(19) United States
(12) Patent Application Publication
(10) Pub. No.: US 2005/0209687 A1
(43) Pub. Date: Sep. 22, 2005

(54) Artificial vessel scaffold and artifical organs therefrom

(75) Inventors: James V. Sitzmann, Potomac, MD (US); Eugene V. Sitzmann, Cooke, IL (US)

Correspondence Address: CROWELL & MORING LLP;INTELLECTUAL PROPERTY GROUP, P.O. BOX 14300, WASHINGTON, DC, 20044-4300, US

(73) Assignee: Bioartis, Inc., Pittsford, NY (US)

(21) Appl. No.: 10/505,131

(22) PCT Filed: Feb. 19, 2003
(86) PCT No.: PCT/US03/04505

§ 371 Date: Apr. 20, 2005

Related U.S. Application Data
(60) Provisional application No. 60/357,118, filed on Feb. 19, 2002.

(51) Int. Cl. A61F 2/06; A61F 2/24; A61M 1/12; C12N 5/08; A61F 2/04

(52) U.S. Cl. 623/1.41; 435/398; 623/23.64; 623/2.13; 623/3.17

(57) ABSTRACT

An artificial vessel scaffold is provided, of biocompatible materials and capable of being coated with selected cell types. A plurality of artificial organs are provided, formed of a biocompatible scaffold material and coated with selected cell types.

### CROSS-REFERENCE TO RELATED APPLICATION

This applications claims benefit of priority of U.S. Provisional Application No. 60/357,118, filed Feb. 19, 2002.

### FIELD OF THE INVENTION

The present invention relates generally to the field of biomaterials, implantable medical devices and cell biology. In particular, the invention relates to an artificial vessel scaffold, methods of making the same, and artificial organs made therefrom. The invention includes the manufacture of artificial organs and vessels, resulting in various devices for sustained growth of species specific (human, animal) cells, for species specific, medically suitable purposes. For example, biomaterial devices according to the invention include, but are not limited to transplantable organs, such as blood vessels, liver, bone, tendon/ligaments, and/or skin; transplantable endocrine glands, such as thyroid, parathyroid, pancreas, adrenal, pituitary, testis, and/or ovaries. The invention also encompasses transplantable genetically engineered cellular protein delivery.

### BACKGROUND OF THE INVENTION

A native vessel is a complex composite. In its simplest description, an artery is a tube that transports blood through various parts of the body. Vessels include both arteries and veins, each having unique demands. For example, arteries experience high shear forces and pressures as blood is pumped through them. Vessels are pliable, resilient and tough entities that consist of smooth muscle cells, lamina and an inner lining of endothelial cells. Their composition and morphology vary depending on the environment and vessel size and type.

Intense effort has been made for many decades to obtain a permanent artificial vessel that can be fabricated in vitro and has equivalent physiological properties as the native target vessel. No single approach has been completely successful in being able to deliver a product that achieves the performance requirements and the need for rapid fabrication. Veins and bioartificial veins have been employed to replace arteries, but they lack the strength and durability needed for general use in the high shear environment of the arteries.

Ideally, it is desired to mimic the design and thus the function of the native vessel as closely as possible. To this end, bovine collagen has been used as a scaffold matrix material upon which vascular smooth muscle cells and endothelial cells are grown. This approach is being developed for carotid artery replacements. The disadvantage to this approach is that bovine collagen is not the most suitable material, since it is expensive and it must be harvested from an animal, and may precipitate immune response inflammation.

The idea of a synthetic scaffold, as a base material for vascular implants, is powerful in its simplicity and matches most closely what nature uses. However, attempts to create synthetic scaffolds, for example from porous polymers, often prove to be too rigid, leading to hyperplasia and increased thrombosis frequencies over time. Questions of optimal morphology and flexural/tensile properties are issues whose solutions are not obvious.

Current technologies have also employed multi-filament fibers that are woven together. However, tubing made from this approach lacks control of micro-porosity (uniform size and spacing) needed for optimal cell entrainment, and also lacks the flexibility and toughness achievable with high porosities.

Accordingly, the synthetic scaffolds of the prior art fail to achieve the optimal morphology and flexural/tensile properties of natural vessels. Therefore, there exists a need for a synthetic scaffold which has compatible physiological properties to a natural vessel. Likewise, specific cells must be able to intercalate into the polymer scaffold. Intercalation builds up wall thickness using cells, and eliminates direct blood contact with the matrix under high shear conditions. High shear contact with noncellular material leads to adverse long term (greater than months) effects.

In addition to the shortcomings presently felt in the synthetic vessel art, there are similar deficiencies in presently-available synthetic organs. While the demand for replacement organs, particularly in humans, has far outpaced available transplant organs, synthetic devices have met with limited success and use. Compatibility remains an issue, as well as the concern regarding contamination and infection, especially for xenotransplantation. Thus, it would be of great use in the art to have biocompatible synthetic organs that could be used as replacement organs, in addition to existing organs, or as temporary devices used while a patient awaits a permanent substitute or gains strength for a procedure inserting the same. Finally, such biocompatible organs must be capable of rapid time-to-harvest. Current technologies require from several weeks to many months to form a fully functional device. In the transplant organ field, this delay may be fatal.

### SUMMARY OF THE INVENTION

One object of the present invention is to provide a synthetic scaffold that is durable yet flexible and will withstand wear and tear of use but be biocompatable.

A further object of the invention is to provide replacement organs for placement in a host, and methods and devices useful in making the same. A host is any mammal, including but not limited to humans.

According to a first embodiment of the present invention, an artificial vessel scaffold is provided, comprising a plurality of elongated scaffold panels arranged in laterally abutting relation to form a tubular structure, and a plurality of circular fibers, wherein the elongated scaffold panels each comprise a first parallel strand of fiber and a second parallel strand of fiber fixedly connected by a plurality of connection fibers oriented substantially perpendicular to the first parallel strand and the second parallel strand, wherein the circular fibers encircle and are fixedly connected to the tubular structure, and wherein the tubular structure defines an inner diameter and an outer diameter. Optionally, this embodiment can further comprise a layer of endothelial cells attached to the internal diameter of the artificial vessel scaffold and/or a layer of smooth muscle cells attached to the outer diameter of the artificial vessel scaffold. The inner diameter of the artificial vessel scaffold of this embodiment may be from 0.5 to 3.0 cm. Optionally, this embodiment may further comprise a layer of digestible material within the internal diameter and attached to the artificial vessel scaffold.

According to a further embodiment of the present invention, a cellular growth chamber is provided, comprising a vessel, an opening in the vessel that allows insertion and removal of a vessel scaffold and that is sealingly closeable, a sealable port providing a opening in the vessel and allowing inlet and outlet of cell culture solution, and an environmental control capable of monitoring environmental conditions within the vessel. Optionally, the sealable port may comprise a first port with an inlet tube and a second port with an outlet tube. The environmental control of this embodiment may also provides adjustment of environmental conditions for favorable cell growth conditions.

According to a further embodiment of the present invention, an artificial vessel scaffold coated with cellular material in a cellular growth chamber according the preceding embodiment is provided. A plurality of such coated artificial vessel scaffolds may comprise an artificial organ.

According to a further embodiment of the present invention, an artificial liver is provided, comprising a common entry region comprised of a hollow cylindrical tube and having a first end and a second end, at least four individual entry regions each having a first end and a second end, the first end of the individual entry regions being fixedly connected to the second end of the common entry, at least four inner vessels, the inner vessels having a first end and a second end, the first end of the inner vessels being fixedly connected to the second end of the individual entry regions, at least four individual exit regions having a first end and a second end, the first end of the individual exit portions being fixedly connected to the second end of the inner vessels, and a common exit region comprised of a hollow cylindrical tube and having a first end and a second end, the first end of the common exit region being fixedly connected to the second end of the individual exit regions, wherein the first end of the common entry portion and the second end of the common exit portions are fixedly connected to a patient, wherein the individual entry regions, the inner vessels, and the individual exit regions are comprised of an artificial scaffold having an inner surface and an outer surface, wherein the inner surface of the artificial scaffold is coated with vascular endothelial cells, and wherein the outer surface of the artificial scaffold is coated with hepatocytes.

According to a further embodiment of the present invention, an internal or external artificial liver is provided, comprising an artificial liver according to the preceding embodiment located within a waterproof container having a first end and a second end, a first pump located at the first end of the container, and a second pump located at the second end of the container, wherein the first end of the waterproof container is connected to and in liquid communication with a patient's artery, and wherein the second end of the waterproof container is connected to and in liquid communication with the patient's vein.

According to a further embodiment of the present invention, an artificial pancreas is provided, comprising at least two artificial pancreas units, each unit comprising a cylindrical mainline scaffold and a plurality of side branches and having a first end and a second end, a first connection tube having a first end fixedly connected to the first end of the artificial pancreas units and having a second end fixedly connected to and in fluid communication with a patient, and a second connection tube having a first end fixedly connected to the second end of the artificial pancreas units and having a second end fixedly connected to and in fluid communication with a patient, wherein the mainline scaffold has a first end and a second end, wherein the side branches have a first end and a second end, wherein the first end of the side branches are fixedly connected and in fluid communication with the mainline scaffold, wherein the second end of the side branches are fixedly connected and in fluid communication with the mainline scaffold at a point on the mainline scaffold closer to the second end of the mainline scaffold than the first end of the mainline scaffold, and wherein the mainline scaffold and the side branches are coated with hormone producing islet cells.

According to a further embodiment of the present invention, an artificial heart valve is provided, comprising a circular ring, and a plurality of leaflets each having two generally parallel generally flat surfaces and an edge around the perimeter of the leaflets, wherein a first portion of the leaflet edge is fixedly and flexibly connected to the circular ring, wherein the leaflet is sized and shaped so a second portion of the leaflet edge opposite the first portion of the leaflet edge is located approximately in the center of the circular ring, and wherein the circular ring and the leaflets are comprised of a scaffold according to the first embodiment of the present invention.

According to a further embodiment of the present invention, an artificial cardiac ventricle is provided, comprising a hollow generally cylindrical central region having a bottom and an apex, the bottom being wider than the apex, and a hemispheric base region fixedly connected to the bottom of the central region, wherein the central region and the base region are comprised of a scaffold according to the first embodiment of the present invention. Optionally, this embodiment may further comprise a jacket enveloping the artificial cardiac ventricle and fixedly attached to the apex of the central region.

According to a further embodiment of the present invention, a cardiac pump is provided, comprising a motor in communication with a pumping means, and a generally cylindrical compressible cardiac replacement unit, wherein the pumping means is comprised of at least one wheel capable of rolling along the length of the cardiac replacement unit to compress and decompress the cardiac replacement unit.

According to a further embodiment of the present invention, a cardiac pump is provided, comprising a motor in communication with a pumping means, and a generally cylindrical compressible cardiac replacement unit located inside a fluid displacement unit and in fluid communication with at least one tube leading outside the fluid displacement unit, wherein the pumping means is comprised of a fluid displacement device, the fluid displacement device being capable of increasing and decreasing the pressure of fluid within the fluid containment unit, and wherein the increasing and decreasing pressure of fluid causes compression and decompression of the cardiac replacement unit.

According to a further embodiment of the present invention, an artificial cardiac device is provided, comprising two artificial cardiac ventricles, each comprising a hollow generally cylindrical central region having a bottom and an apex, the bottom being wider than the apex, a hemispheric base region fixedly connected to the bottom of the central region, and at least one cardiac pump as described above, wherein the at least one pump compresses and decompresses the two artificial cardiac ventricles. The device may further comprise a biocompatible housing located around the outer boundaries of the cardiac device, wherein the biocompatible housing containing the cardiac device is located inside a patient.

The present invention involves a novel scaffold, which is reproducibly made and easily tailored to specific needs, and is fabricated, for example, using porous membrane tubing. The porosity is made and controlled by selective solubilities of the components of the tubing. In one embodiment of the invention, co-extruded or cast tubing can be made from two or more polymers, wherein one of the polymers and/or other material in the tubing is dissolved away upon exposure to solvent. The design allows initial growth of cells in a sterile laboratory setting, with subsequent implantation of the device with viable, functional cells into a host recipient. To date, implantation has been accomplished with similar devices in multiple animals of each cell type (see citations below). This will allow the use of the inventive devices and organs as functional replacement of blood vessels, and other listed organs, for medically indicated purposes in mammals, and in humans.

According to one embodiment of the present invention, porous polymeric material is fabricated as a scaffold upon which cells are then grown, to form durable, optionally elastic, non-thrombogenic devices. The polymeric material may be a porous thermoplastic tube, made by extrusion or molding processes of an appropriate blend of water-insoluble polymers (such as nylon-11, thermoplastic urethane, polysiloxanes, and combinations thereof in various ratios) and water-soluble polymers (such as polyethylene oxides). The tube wall thickness and diameter can be varied by the appropriate choice of extruder dies or molds. A wall thickness of 50 microns is nearly ideal for vascular assemblies. Tubes of various diameters can be made by extruding a formulation comprising a water-insoluble polymer, such as nylon-11, and a water-soluble polymer, such as polyethylene oxide.

Suitable water-insoluble polymers which can be used in the fabrication of the porous tubes according to the invention include nylons, such as nylon-11, thermoplastic polyurethanes, polysiloxanes, and combinations thereof. The insoluble polymer can be a high molecular weight thermoplastic or thermoset resin. The insoluble polymer or resin preferably has sufficient polarity to provide good adhesion to cells. It also must be autoclavable or sterilizable and capable of providing good durability and flexibility. Specific examples of useful, water-insoluble polymers useful in the invention include:

| Product Code | Description | CAS Registry No. |
|---|---|---|
| ORGASOL 1001 UD NAT | Nylon 12 | [25038-74-8] |
| ORGASOL 1002 D Natural | Nylon 6 | [25038-54-4] |
| ORGOSOL 2001 UD NAT | Nylon 12 | [25038-74-8] |
| ORGOSOL 2001 UD NAT | Nylon 12 | [25038-74-8] |
| ORGOSOL 2002 D NAT | Nylon 12 | [25038-74-8] |

Nylon-11 is particularly useful as a matrix material and in bioartifical organ fabrication, because of its non-swelling properties compared to other nylons. It is autoclavable, hydrolytically resistant, water-insoluble, and durable. Combinations of nylon-11 and thermoplastic urethanes (TPU's) and/or TPU as the water-insoluble polymer are also useful to enhance the final flexibility of the porous tubing. The discovery of this type of porous tubing, which can be used as a matrix scaffold onto which and into which human cells can be grown, has led to a whole new arena of bioreactive devices.

The extruded tube is then immersed in an appropriate solvent to extract some or all of the water-soluble polymer(s), leaving a porous, optionally pliable, thermomechanically tough tubing. The pores in the nylon tubing were thus generated as a result of the water extraction of the polyethylene oxide. By varying the ratio of water-insoluble polymer to water-soluble polymer, the porosity of the tubing can be vaired from about 50% to about 80% and the average pore size from about 0.5 micron to about 5 microns.

For more flexible and rubber-like porous materials, part or all of the nylon can be substituted with a thermoplastic urethane.

The polymer matrix that dissolves away is preferably a medium molecular weight nonionic thermoplastic, such as polyethylene oxide (PEO).

The resulting tubing does not contain plasticizers, solvents, or other undesirable or harmful ingredients. The porosity of the tubing may range from approximately 50 to 80%, and is controlled by the ratio of the water-insoluble polymer to the water-soluble polymer. Pore size is controllable between approximately 0.5 micron to 5 micron. Typical tubes had a 75% porosity with an average pore size of 2 microns and a wall thickness of 50 microns.

After the porous polymer scaffold has been prepared, it is then intercalated with specific desired cells. This is achieved by using a bioreactor to selectively grow desired cells on the matrix to form an artificial organ. For example, an artificial artery may be produced by growing smooth muscle cells on the exterior and endothelial cells on the interior of the porous tube. The process builds up wall thickness using cells and eliminates direct blood contact with the matrix under high shear conditions. The presence of a smooth layer of natural cells on the inside of the artificial vessel is important because over the course of several months, high shear contact with noncellular material leads to adverse long term effects.

Cells enter the pores of the matrix and interconnect to form a uniform and continuous layer outside of the polymer tube. The cells are locked into the matrix because of the pore structure permits intimate cell-cell contact through the inner and outer walls of the tubing. The polar amide moieties of the porous polymer, which are at relatively low densities, serve as contact points for cellular adhesion. The uniform cell coverage isolates the polymer tube from the host environment, further enhancing the device's non-thrombogenicity. The small pore size and high porosity of the matrix permits improved cell-cell contact, which leads to enhanced in vivo response to environmental stimuli.

By growing cells from suitable various organs or a suitable porous polymer matrix, it is possible to produce synthetic organs which can be transplanted into a patient to replace or augment failing natural organs. Candidate transplantable synthetic organs include blood vessels, liver, bone, tendons/ligaments, skin, and others. Similarly, by growing cells from various endocrine glands on a suitable porous polymer matrix it is possible to produce transplantable synthetic endocrine glands. Synthetic glands which may be produced in this way include thyroid, parathyroid, pancreas, adrenal, pituitary, testis and ovaries. It is also possible by growing cells which have been genetically engineered to produce various cellular proteins or enzymes on suitable porous polymeric matrices to produce implants which will selectively secrete the desired protein or enzyme after implantation in a patient.

The invention can thus produce a variety of synthetic devices which can be implanted in a patient to facilitate sustained growth of species specific (i.e., human or animal) cells to provide a desired, species specific, medically applicable treatment, such as an artificial organ.

By combining grown cells with a flexible, tough, and highly porous polymer tube leads to a device that can be used as a replacement for arteries. Resulting devices can also be modified by a change in the type of cells grown onto/into it. For example, appropriate cell selection can provide a functional bioartificial liver. Additionally, modification of the polymer matrix with inorganic fillers can provide a scaffold upon which cells are grown to produce a device suitable as artificial bone or cartilage.

Other objects, advantages and novel features of the present invention will become apparent from the following detailed description.

### BRIEF DESCRIPTION OF THE DRAWINGS

FIG. 1A shows a single scaffold panel;

FIG. 1B shows a double scaffold panel;

FIG. 2A shows a perspective drawing of a pentagonal scaffold;

FIG. 2B is a cross-sectional view of a pentagonal scaffold;

FIG. 2C is a cross-sectional view of a octagonal scaffold;

FIG. 3 is a cut away view of a scaffold cell growth chamber;

FIG. 4 is a perspective view of a scaffold with tube lining;

FIG. 5 is a perspective view of a scaffold with cellular coatings;

FIG. 6A schematically represents the scaffold arrangement of an internal artificial hepatic organ;

FIG. 6B shows a cross sectional view at point I-I of FIG. 6A;

FIG. 6C schematically represents the scaffold arrangement of an internal artificial hepatic organ;

FIG. 6D shows a perspective view of a portion of an internal artificial hepatic organ;

FIG. 7A is a schematic drawing of common and individual entry or exit regions of an internal artificial hepatic organ;

FIG. 7B is a schematic drawing of common and individual entry or exit regions of an internal artificial hepatic organ;

FIG. 7C is a schematic drawing of common and individual entry or exit regions of an internal artificial hepatic organ;

FIG. 8 is a cut away view of a hepatic cell growth chamber;

FIG. 9 is a schematic drawing of the configuration of an external artificial hepatic organ;

FIG. 10A is a schematic drawing of an artificial pancreas;

FIG. 10B is a detailed view of a portion of an artificial pancreas;

FIG. 11A schematically represents scaffold arrangement in an artificial pancreas;

FIG. 11B schematically represents scaffold arrangement in an artificial pancreas;

FIG. 12A is a cross sectional view of an entry or exit portion of an artificial pancreas;

FIG. 12B is a perspective view of an entry or exit portion of an artificial pancreas;

FIG. 13 is a cut away view of a pancreatic cell growth chamber;

FIG. 14A is a top view of an artificial heart valve;

FIG. 14B is a side view of an artificial heart valve;

FIG. 15 is a cut away view of a heart valve growth chamber;

FIG. 16A is a perspective view of a cardiac replacement compartment;

FIG. 16B is an additional perspective view of a cardiac replacement compartment;

FIG. 16C is a side view a cardiac replacement compartment with a jacket;

FIG. 17 is a side view of a valve and extender region;

FIG. 18 is a cut away view of a cardiac cell growth chamber;

FIG. 19 schematically represents an internal cardiac pump with roller wheels;

FIG. 20 schematically represents an internal cardiac pump with fluid;

FIG. 21A is a schematic drawing of a component of an internal cardiac pump;

FIG. 21B shows an enlarged view of a dual flywheel for use on an internal cardiac pump;

FIG. 21C is a schematic drawing of a component of an internal cardiac pump;

FIG. 22 is a schematic representation of an internal cardiac replacement system;

FIG. 23A schematically shows an external cardiac pump;

FIG. 23B is a top view of an external cardiac pump; and

FIG. 23C shows an enlarged view of the roller portion of an external cardiac pump.

FIG. 24 is a schematic illustration of a synthetic artery according to the invention.

### DETAILED DESCRIPTION OF EMBODIMENTS ACCORDING TO THE INVENTION

The present invention may be understood more readily by reference to the following detailed description of particular embodiments of the invention and the specific examples. The terminology used herein is for the purpose of describing particular embodiments only and is not intended to be limiting.

### EXAMPLE I

### Fabrication of an Artificial Vessel Scaffold

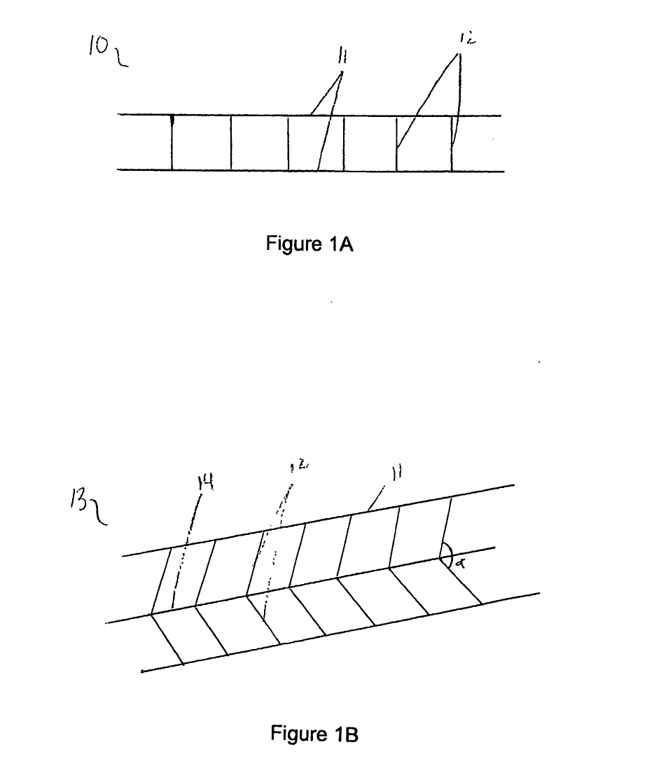

As depicted in FIG. 1A, a panel 10 is formed by connecting two or more, preferably two or three, parallel strands 11 of nonabsorbable material, preferably non-immunogenic, with minimal elastic potential, for example, #10 nylon fibers, with shorter, perpendicular strands 12 of the same diameter material. Perpendicular strands 12 are spaced approximately 0.5 to 1.0 μM apart and are oriented at approximately 90° to parallel strands 11. All strands are fixedly connected to one another by means appropriate for the selected material. Such means include, but are not limited to, heat and adhesive. For example, high temperatures induce the binding capability and anneal certain fibers such as SILASTIC™, or microscopic amounts of sealant substances such as silicone, polyurethane, or polyethylene can permanently join such fibers as would be used in the present invention.

In the embodiment illustrated in FIG. 1A, the panel is shown as having a relatively flat shape. Flat panels may offer advantages such as ease of fabrication. Optionally, as shown in FIG. 1B, a double panel 13 can be fabricated with a common longitudinal strand 14 and with perpendicular strands 12 arising at desired intervals. Perpendicular strands 12 connect common longitudinal strand 14 with one or two parallel strands 11. Perpendicular strands 12 may attach on opposite sides of common longitudinal strand 14, as shown in FIG. 1B, optionally, they may alternate along the length of common longitudinal strand 14 with an approximately equal number of perpendicular strands 12. FIG. 1B shows each of the perpendicular strands 12 connecting common longitudinal strand 14 with one parallel strand 11 so that an angle a is formed along common longitudinal strand 14. Additional parallel strands 1 may be present between common longitudinal strand 14 and the furthest parallel strand 11. This may increase strength, particularly in larger scaffolds. A further option is to prepare a triple panel (not shown). While the panels depicted are generally flat or angular, it is within the scope of the invention to utilize panels that have a curved conformation, thereby better approximating the generally round configuration of natural vessels.

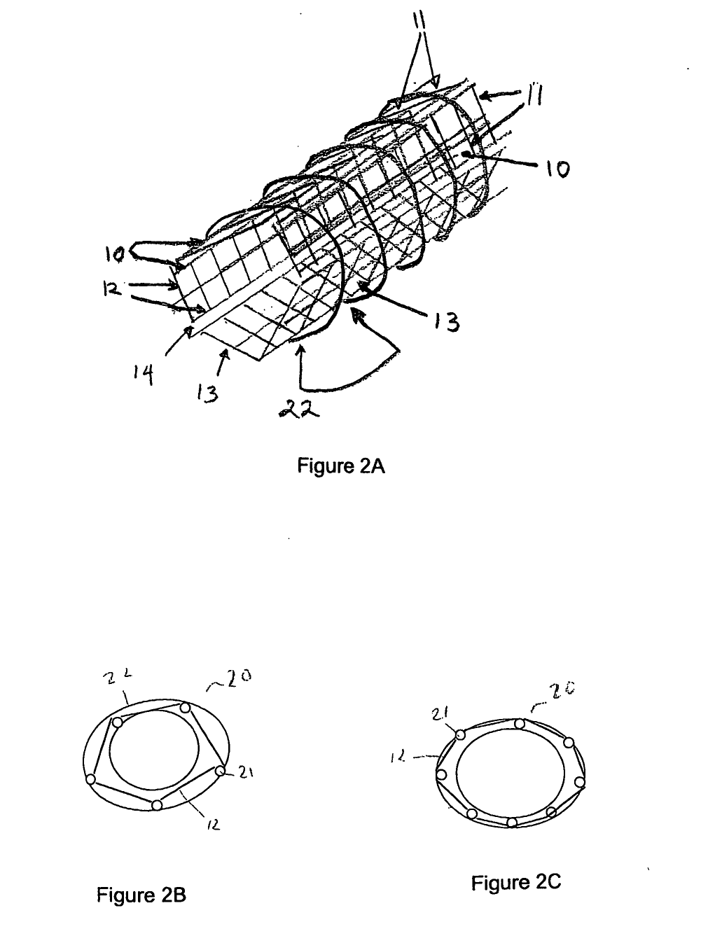

As shown in FIG. 2A, panels 10 and/or double panels 13 are prepared and arranged so that parallel strands 11 of each panel 10 or double panel 13 are parallel to one another forming a roughly cylindrical scaffold 20. Panels 10 or double panels 13 are arranged with similar angles between each pair of panels, approximately 25° to 45°, for example, 25°, 30°, 35°, 40°, or 45°. A gap 21 exists between each set of panels 10 or double panels 13 excepting the spaces where the panels are connected to one another. Gap 21 measures approximately 0.5 μM to 1 μM.

To hold panels in a generally cylindrical form, abutting longitudinal edges of adjacent panels are connected at spots spaced along the panel length. The intervening unconnected lengths allow the panels of the resulting tubular structure to flex and shift relative to each other in response to pressure fluctuations within the vessel. This, in turn, enables the synthetic vessels to stretch slightly under pressure much like natural vessels and increases their durability and reliability. It is particularly advantageous to stagger the connection spots on opposite sides of each panel in order to avoid forming unstretchable nodes along the length of the vessel.

A circumferential ring 22 made of SILASTIC™ (Dow Coming, Midland, Mich.) or other suitable elastic, preferably non-antigenic, material fiber is attached to the outer surface of the panels 10, 13 to anchor the cylindrical panel arrangement. A plurality of rings 22 are spaced at intervals 100 μM to 600 μM apart. Decisions regarding inner and outer diameter, length, etc., would be determined based on designed vessel usage. Measurements correspond to natural vessels and vary with intended use.

During fabrication, panels 10, 13 are oriented around a common core (not shown), which may be tube or funnel shaped and made of a smooth, hard material. The use of a core facilitates building of scaffold 20 and may be simply, rapidly removed after scaffold 20 completion. After placing panels 10, 13 around the core, they are held in position with rings 22, which can be bound to the fibers of panels 10, 13 with any means preferred for the materials selected. The number of panels 10, 13 selected varies with desired use, two examples of cross sectional shapes for scaffold 20 include a pentagon as shown in FIG. 1D, and an octagon, as shown in FIG. 1E.

Rings 22 could be made from a number of materials known in the art, including, but not limited to, SILASTIC™, methacrylate, nylon, dacron, polydimethylsiloxane, polyurethane, polyethylene, vicryl, gore-tex, latex, rubber, elastic, glass, ceramic, and plastic. The strands used in panels 10, 13 could be made from any number of materials known in the art, including, but not limited to, SILASTIC™, methacrylate, nylon, dacron, polydimethylsiloxane, polyurethane, polyethylene, vicryl, gore-tex, polyproplene, octyl-2-cyanoacrylate, polymethylacrylate, polyactide, aramid, polystyrene, poly L-lysine, aluminum, copper, stainless steel, and titanium.

Materials appropriate for the core that is helpful in fabrication of scaffold 20 include latex, rubber, elastic, glass, ceramic, plastic, aluminum, copper, stainless steel, and titanium.

### EXAMPLE II

### Fabrication of a Bioartificial Vascular Device

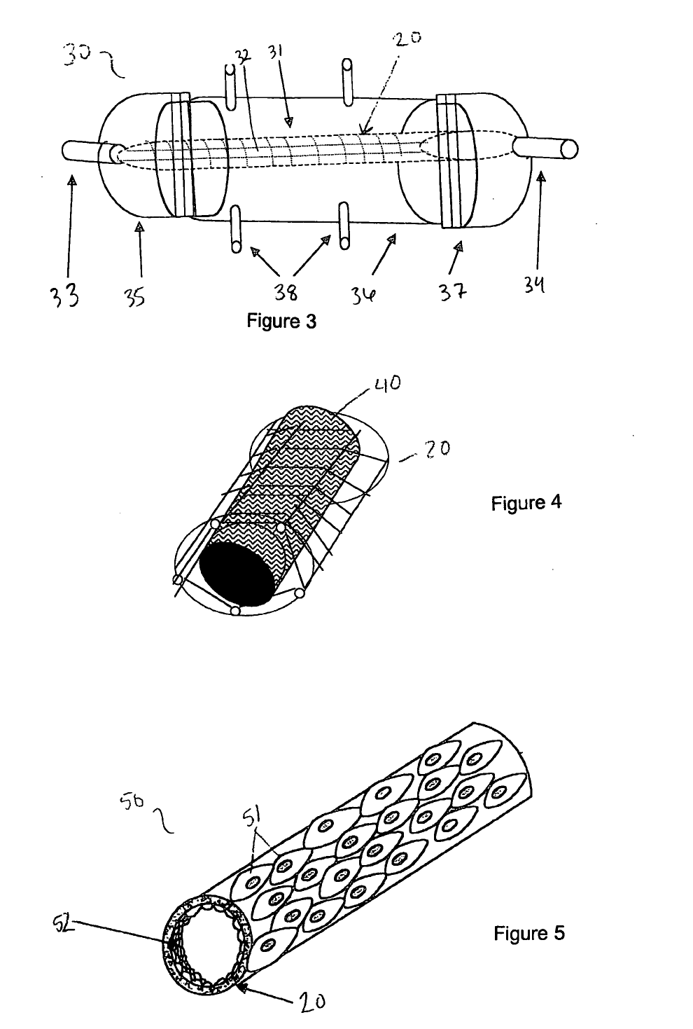

Prior to implanting a prosthetic vessel as described above, vascular endothelial cells and vascular smooth muscle cells must be grown upon a permanent structure. This process begins with fabrication of an artificial vessel scaffold, optionally scaffold 20 according to Example I. A layer of vascular smooth muscle cells is grown along the outer surface of scaffold 20. A layer of endothelial cells is grown along the inner surface of scaffold 20. The addition of cellular layers can be accomplished by placing scaffold 20 in a vascular cell growth chamber 30, as shown in FIG. 3, so that scaffold 20 forms an outer chamber 31 and an inner chamber 32 of vascular cell growth chamber 30. Outer chamber 31 is filled with a cell culture solution containing vascular smooth muscle cells and incubated to allow the cells to attach to the outer surface of scaffold 20, approximately two days.

Following the deposit of an outer layer of cells, a cell culture solution containing endothelial cells is flowed through inner chamber 32. This is accomplished by pumping the solution in a first port 33 of vascular cell growth chamber 30 and out a second port 34 of vascular cell growth chamber 30 to allow the endothelial cells to attach to the inner surface of scaffold 20 and to properly align. First port 33 and second port 34 should correspond in diameter to scaffold 20. The solution containing endothelial cells may be flowed through scaffold 20 for sufficient time, for example, two weeks. Following the deposit of both cellular layers, scaffold 20 is removed from vascular cell growth chamber 30 and implanted immediately, or stored at 0-30° C. for 20-36 hours.

This type of cellular scaffold coating has been previsouly described (202). The smooth muscle cells are allowed to cover the strands that form the scaffold. Additional cells deposit and grow forming a web, then a solid tube of smooth muscle cells over, around, and in between the scaffold structure. The subsequent flow of endothelial cell culture through the coated scaffold serves to form a lining to the artificial vessel as well as provide a continuous opening along the center of the scaffold. This procedure most nearly represents the cell growth that occurs in a native vessel and, when performed in conjunction with the novel scaffold herein described, forms an artificial vessel with properties remarkably similar to a native vessel.

Other features of the presently described vascular cell growth chamber 30 include separate portions, such as a first end 35, a middle portion 36, and a second end 37. Side ports 38 are provided on middle portion 36 to allow for introduction and removal of the vascular smooth muscle cell solution. The separate portions may be connected by a watertight thread or other appropriate means. It may be preferable to construct vascular cell growth chamber 30 of a clear plastic material in a cylindrical shape. Preferred sizes for vascular cell growth chamber 30 are a length of 8 to 20 cm and a diameter of 0.6 to 4.0 cm. It is understood that the present invention encompasses equivalent chambers that are known and those that will be developed in the art.

If usage indicates cellular transmigration or movement through the gaps in scaffold 20, an inner tube 40, as shown in FIG. 4, may be constructed and placed along the inner surface of scaffold 20. Inner tube 40 could be constructed of a digestible fiber such as vicryl, a carbohydrate or polyglycic matter to be resorbable. Inner tube 40 may also be used to prevent compression or collapse of scaffold 20 after implantation. Inner tube 40 may have a high distribution or incidence of consistent pore size, from 50 to 150 mM, for example 100 mM, in diameter.

As shown in FIG. 5, scaffold 20 has become a coated scaffold 50 with an outer layer 51 and an inner layer 52 of cells. Based on the size of scaffold 20 used and the thickness of outer layer 51 and inner layer 52, a plurality of coated scaffold 50 or artificial vascular device sizes may be accomplished. An inner diameter is defined by the cells grown on the inner surface of scaffold 20. Dimensions such as inner and outer diameter and length will necessarily vary depending on the intended use of the artificial vascular device.

Applications of the present invention include coronary or cardiac arterial vessels, arterial venous fistula, larger (i.e., for abdominal organs such as liver or kidney, cranial directed, or upper extremity based) arterial and larger venous replacement. For coronary artery replacements and/or grafts, the preferred inner diameter is 350 μM to 1000 μm with an external diameter of 500 μm to 1200 μm. A wall thickness of 200 μm to 300 μm and a length from 9.6 to 12 cm are preferred.

For arterial venous fistula, useful for subcutaneous placement to provide transcutaneous access for dialysis service (i.e., artificial renal support, artificial nutrition, or artificial hepatic support) the graft preferably has an internal diameter from 1 to 3 cm, an external diameter of 1.5 to 3.5 cm, a wall thickness of 250 to 400 mm, and a length of 12 to 20 cm.

Major artery replacement uses would include grafts for replacement of carotid, upper extremity (axillary artery or brachial artery), or abdominal uses; with an inner diameter from 2 to 2.5 cm, an external diameter from 2.5 to 3 cm, a wall thickness from 500 to 600 mm, and a length of 10 to 15 cm.

Major vein replacements could be accomplished with grafts for replacement of carotid, upper extremity (axillary vein or brachial vein), abdominal organ or portal vein uses. The inner diameter of such a configuration should be approximately 2 to 3.5 cm with an external diameter from 2.5 to 4 cm and a wall thickness from 400 μm to 500 μm. Length could be elected based on specific use. Approximate lengths are from 5 to 10 cm.

Vascular “artifact” or potential prostheses range from narrow internal diameter, useful for arterial-venous fistula prosthesis, ranging from 0.75 to 1.10 cm. A very narrow inner diameter of 0.50 to 0.64 cm could be employed in an artificial vessel used for limited arterial vessel replacement for cardiac usage. Such uses include cardiac artery bypass. This would obviate the common practice of harvesting a leg vein, a forearm artery, or an artery harvested from the inner thoracic chest wall. Larger diameter prosthesis ranges having an inner diameter from 1.5 to 3 cm, for example 1.8 cm, could be used as a vascular vessel replacement such as for liver or renal organ transplant. Constructing an artificial vessel with the largest diameter would be useful for upper or lower extremity revascularization.

### EXAMPLE III

### Fabrication of an Internal Bioartificial Hepatic Organ

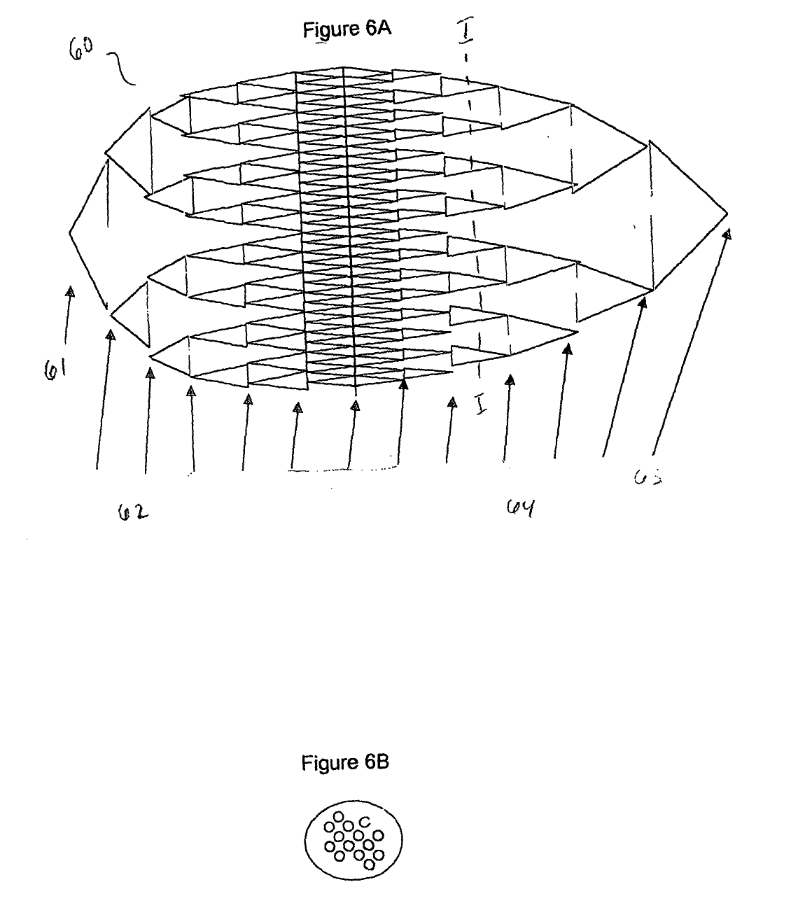

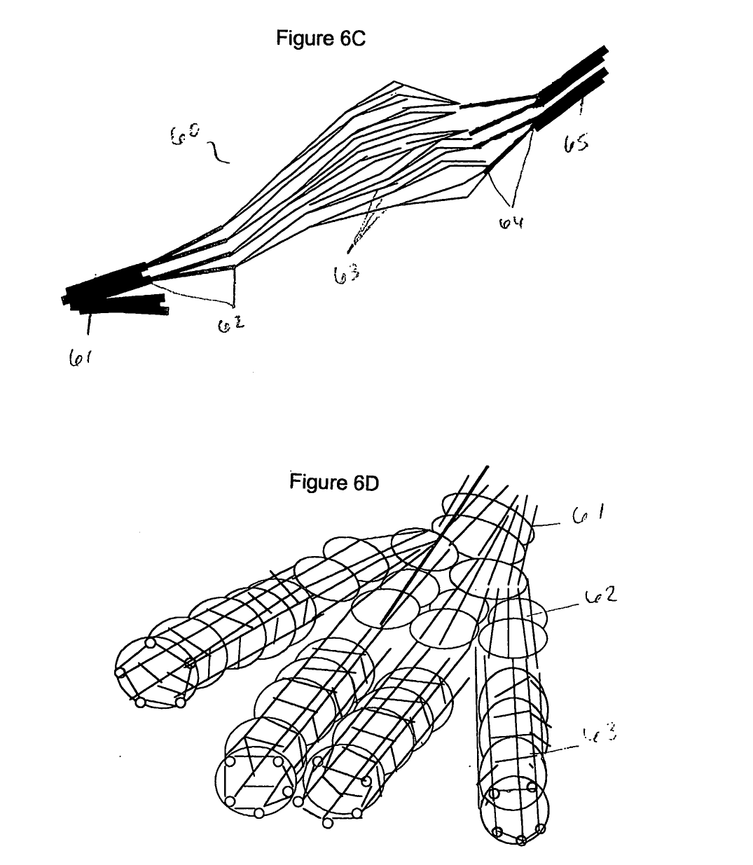

FIG. 6A shows a schematic overview of an artificial internal hepatic organ unit 60. A common entry portion 61 of artificial internal hepatic organ unit 60 is sutured to an abdominal artery (not shown). Common entry portion 61 has an inner diameter of approximately 2 cm and a length of approximately 1 to 2 cm. Extending from the end of common entry portion 61 opposite the artery are a plurality of 2-20 individual entry portions 62. Each individual entry portion 62 has a diameter of about 80-100 μm. Each individual entry portion 62 leads to the first end of a plurality of 4-8 inner vessels 63, each having an inner diameter of about 30-50 μm and a length of 8-12 cm. The plurality of inner vessels 63 originating from one individual entry portion 62 join at their second end to an individual exit portion 64. The individual exit portion 64 has a diameter of about 80-100 μm. Each individual exit portion 64 is joined together at a common exit portion 65 having an inner diameter of approximately 2 cm and a length of 2-4 cm. The common exit portion 65 is sutured to an abdominal vein (not shown). Individual entry portions 62 and individual exit portions 64 are preferably staggered in a branching fashion as shown schematically in FIG. 6A.

The common entry and common exit portions can be made from any suitable tubing material. The remainder of the artificial organ is comprised of synthetic vessels, optionally according to Example I. Connections may be accomplished by, for example, creating a plurality of openings staggered along the length and spaced around the circumference of a common entry tube. A corresponding plurality of first ends of individual entry regions would be placed in liquid communication and forming a seal with the openings in the common entry tube. An alternative example is to form an end region on, for example, the common entry region, that comprises a hemispheric shape with a plurality of openings. First ends of individual entry regions could be placed in each of the openings, in fluid communication and forming a seal with the individual entry regions. Adhesive materials, thermal treatment, and other available methods may be used to join pieces of the organ together.

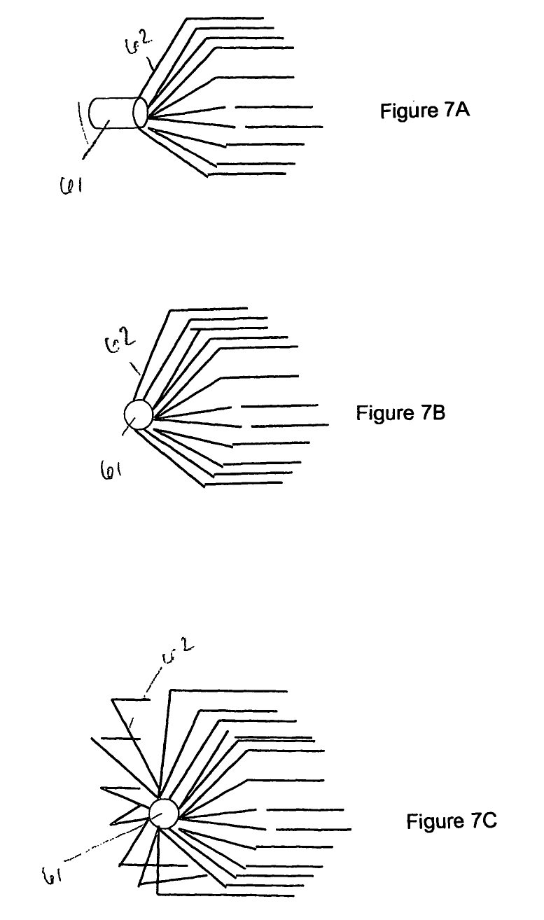

FIG. 6B shows a cross section of artificial internal hepatic organ unit 60 along line I-I. FIG. 6C shows a perspective view of artificial internal hepatic organ unit 60. FIG. 6D shows an enlarged view of a portion of artificial internal hepatic organ unit 60, showing common entry 61 and four individual entries 62 leading to inner vessels 63. In addition to branching individual entry portions shown in FIG. 6, FIGS. 7A, 7B and 7C show alternate arrangements for artificial internal hepatic organ unit 60. FIGS. 7A, 7B and 7C show 10, 12, and 20 individual entry portions 62 arising for a single point of common entry 61, respectively. The multiple individual entry portions 62 shown in FIGS. 7A, 7B, and 7C radiate out in a roughly perpendicular fashion from common entry 61. Corresponding exit regions could be used as well.

For preparation of the artificial internal hepatic organ, a plurality, for example, 10 or 20, of artificial internal hepatic organ units 60 are prepared. Typically, common entry portion 62 and common exit portion 64 comprised of solid fiber construction, allowing for ease of suturing to the abdominal vessels (not shown). The remainder of the artificial internal hepatic organ is comprised of an artificial vessel scaffold, optionally fabricated according to Example I. Each artificial internal hepatic organ unit 60 may be covered in a biocompatible, non-degradable woven material (not shown). Materials include dacron, gore-tex, polyurethane, and polyethylene. This material cover would start at common entry portion 62 and extend to common exit portion 64. Generally, each enclosed artificial internal hepatic organ unit 60 is approximately 10 to 12 cm long and 5 to 8 cm in diameter.

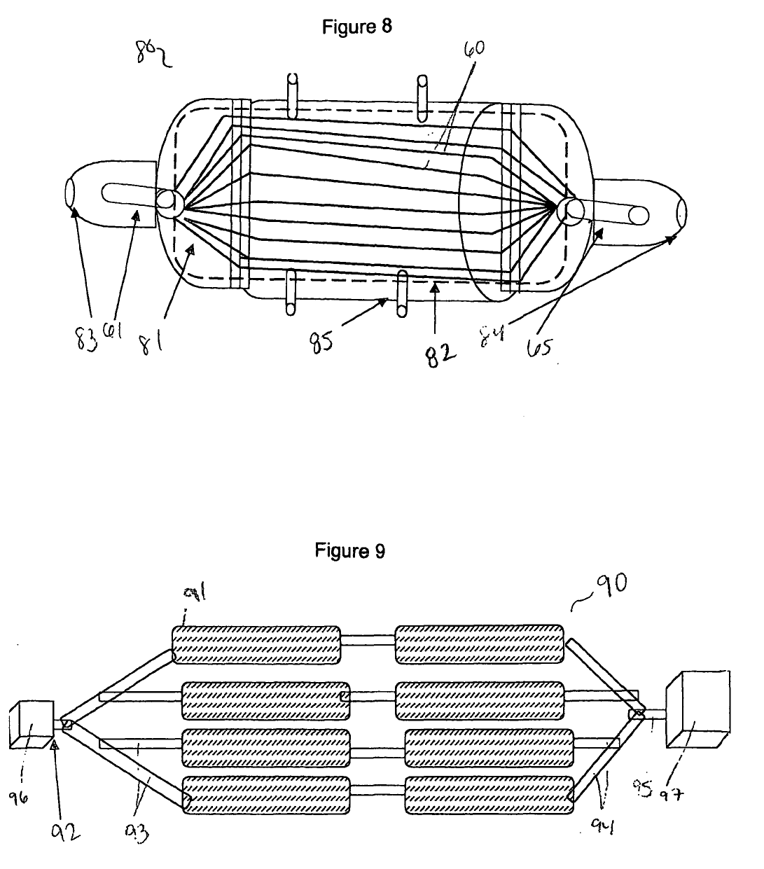

Because the assembled artificial internal hepatic organ comprises scaffolds, it must be coated with cells prior to use of the artificial internal hepatic organ in a patient. A layer of hepatocyte cells is grown along the outer surface of each scaffold in each artificial internal hepatic organ unit 60. A single or multiple layer of endothelial cells is grown along the inner surface of each scaffold as well. One way to accomplish this cellular coating is with the use of a hepatic cell growth chamber 80, as shown in FIG. 8. The assembled artificial internal hepatic organ 81 is enclosed in a nondegradable hepatic jacket 82 and placed in a hepatic cell growth chamber 80. The hepatic cell growth chamber 80 is filled with a cell culture solution containing hepatocytes, incubation follows to allow the cells to attach to the outer surface of each component scaffold, approximately two days.

Following the deposit of an outer layer of cells, a cell culture solution containing endothelial cells is flowed through the artificial internal hepatic organ 81. This is accomplished by pumping the solution in a first port 83 of hepatic cell growth chamber 80 and out a second port 84 of hepatic cell growth chamber 80 to allow the endothelial cells to attach to the inner surface of each scaffold and to properly align. First port 83 and second port 84 should correspond in diameter to common entry portion 61 and common exit portion 65. The solution containing endothelial cells may be flowed through hepatic cell growth chamber 80 for sufficient time. Following the deposit of both cellular layers, artificial internal hepatic organ 81 is removed from vascular cell growth chamber 30 and implanted immediately, or stored at 0-30° C. for 20-36 hours.

Other features of the presently described hepatic cell growth chamber 80 include side ports 85 that allow for addition and removal of the hepatocyte solution. After assembly of artificial internal hepatic organ 81 and cellular coating, finished measurements are approximately 10 to 12 cm length, with a diameter of innermost tubes of 350 to 450 μm, a wall thickness of 100 to 200 μm, and pores in the walls of 0.5 to 1 μm. The space between the tubes and hepatic jacket 82 is approximately 350 to 600 μm, for example, 500 μm.

### EXAMPLE IV

### Fabrication of an External Bioartificial Hepatic Organ

FIG. 9 shows the schematic structure of an external artificial hepatic organ 90. An artificial internal hepatic organ, such as that of Example III, can be contained within synthetic tubing and located externally if desired. Modifications to facilitate external placement include a plurality of enclosures 91 of approximately 2 to 4 cm in diameter and preferably made from clear, plastic tubing. Each enclosure 91 contains an artificial hepatic organ.

A common entry 92 region of external artificial hepatic organ 90 has a diameter of 0.5 to 1.0 cm and connects to individual entry portions 93. Each individual entry portion 93 houses the individual entry of an artificial hepatic organ. Individual entry portions 93 lead to enclosures 91 that house the inner scaffolds of an artificial hepatic organ. Multiple artificial hepatic organs are assembled in series and/or in parallel to provide a desired amount of surface area within external artificial hepatic organ 90. Individual exit portions 94 are provided to allow exit of fluid from each enclosure of external artificial hepatic organ 90. Individual exit portions 94 are connected to common exit portion 95 having a diameter of 0.5 to 1.0 cm. A total of 10 to 20 enclosures 91 may be provided for the external artificial hepatic organ 90.

A first rotary pulsile pump 96 facilitates blood flow through enclosures 91. First rotary pulsile pump 96 is connected to a patient percutaneously through a polyethylene, plastic, SILASTIC™, nylon or other flexible tube via a needle into an artery (not shown) and to common entry portion 92 using appropriate materials. An optional second rotary pulsile pump 97 is provided between and connected to common exit portion 95 and a patient, percutaneously via a needle into a vein (not shown). The pump(s) 96 (97) facilitate blood flow of approximately 100 to 1000 ml/minute at a pressure of 25 to 100 mm Hg through the device and back to the patient.

The bioartificial hepatic organs of the present invention addresse a need in the art to provide a suitable replacement organ for a patient suffering from any form of hepatic deficiency. These patients often are necrotic and highly immunologically challenged. A device made of non-immunogenic materials is desirable for these critical patients. The cellular coatings and underlying non-toxic materials prevent rejection, clotting, or activation of cytokines that could all be abnormally taxing on a patient suffering from hepatic injury, disease, or malfunction.

### EXAMPLE V

### Fabrication of a Bioartificial Pancreas

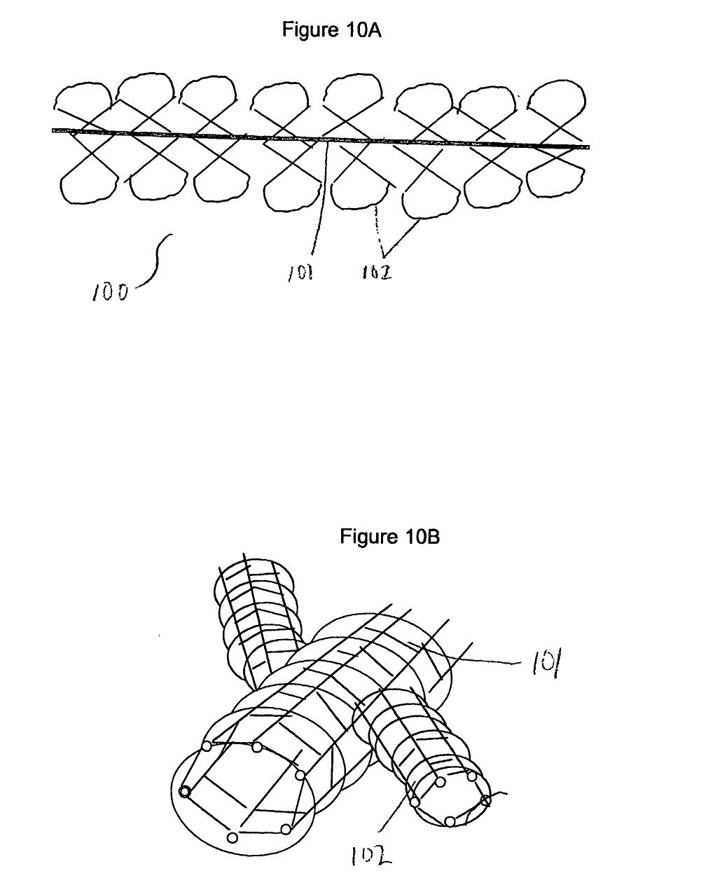

As shown in FIG. 10A, an artificial pancreas unit 100 is comprised of a mainline scaffold 101, a generally cylindrical structure with a diameter of about 0.5 to 1.5 cm and a length of 8 to 24 cm, and multiple side branches 102 with inner diameters of about one quarter to one half that of the mainline scaffold (0.125 to 0.75 cm) and lengths of 7 to 20 cm. The scaffolds may optionally be prepared from materials as described in Example I. Side branches 102 attach at a first end to mainline scaffold 101 and extend contralaterally therefrom. A loop forms along side branch 102 before it reattaches to mainline scaffold 101 at a second end. Multiple side branches 102 in the range of 10 to 20, for example, 16, are provided on mainline scaffold 101. A close up perspective view of one portion of an artificial pancreas unit 100 showing the mainline scaffold 101 and side branches 102 is depicted in FIG. 10B.

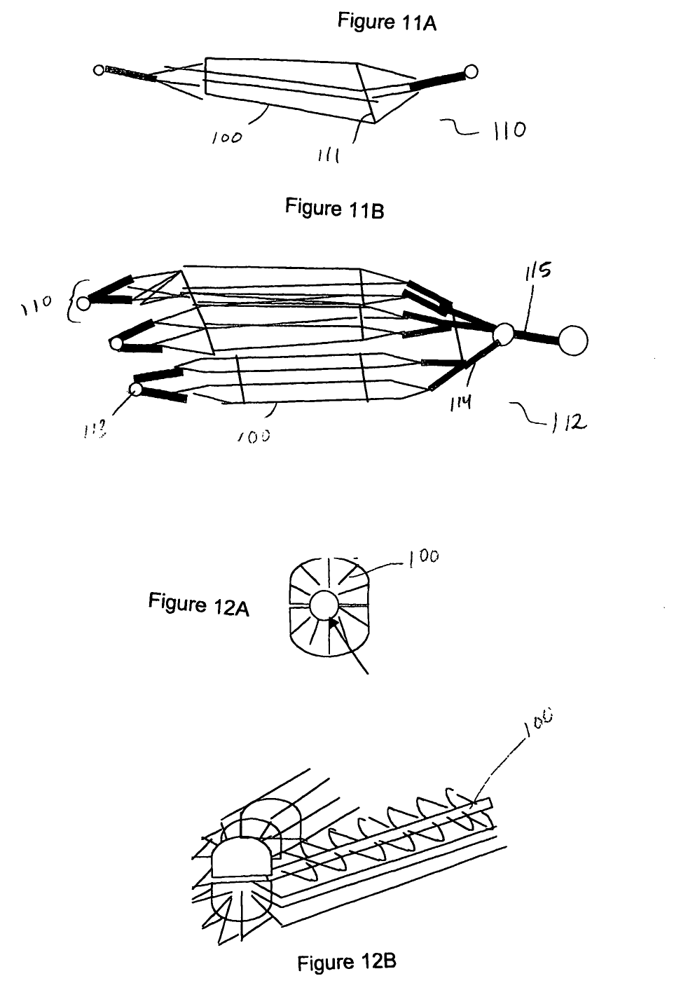

FIGS. 11A and 11B omit representations of side branches 102 for clarity. FIG. 11A depicts the joining of four artificial pancreas units 100 to form a first branching stage 110 of a bioartificial pancreas (entire organ not shown). An optional spacer 111 may be provided which is connected to some or all of artificial pancreas units 100 to maintain a desired spacing among and between artificial pancreas units 100. As further shown in FIG. 11B, three first branching stages 110 can be combined to form a third branching stage 112. As further first branching stages 110 are added, an artificial pancreas 111 is formed according to the desired specifications. The exact number of branching stages utilized will vary, depending upon condition of the patient, proposed insertion location, and other factors.

The artificial pancreas is provided with a common entry region 2 to 5 cm in diameter and is 7 to 20 cm long. The artificial pancreas is comprised of a plurality of artificial pancreas units 100 grouped into branching stages. There the entry portion of each first branching stage 110 is joined in a common entry region (not shown). From the common entry region sprout multiple individual entry regions 113 that lead into each first branching stage 110. At the opposite end, artificial pancreas units 100 converge to an individual exit portion 114 of each branching stage. Individual exit portions 114 join together at a common exit portion, shown as feature 115 of FIG. 11B, it being understood that the artificial pancreas could consist of fewer or more than three branching stages. Common exit portion 115 is similar in size to the common entry portion. FIG. 11B depicts 12 connected artificial pancreas units 100, approximately 2 to 12 artificial pancreas units 100 could be used in one artificial pancreas according to the present invention.

One way to connect multiple artificial pancreas units 100 to a common entry or exit is to connect each artificial pancreas unit 100 as an offshoot radiating outward from the entry or exit at a particular angle. For example, eight mainline scaffolds could be connected, each at an angle of 45 degrees to one another. That is, with a cylindrical entry or exit regions, artificial pancreas units 100 could branch off at 0, 45, 90, 135, 180, 225, 270, and 315 degrees. Twelve artificial pancreas units 100 could be arranged with 30 degrees between each, that is, branches at 0, 30, 60, 90, 120, 150, 180, 210, 240, 270, 300, and 330 degrees. This configuration is shown in cross sectional view in FIG. 12A. A perspective view of a sixteen offshoot configuration is shown in FIG. 12B. Eighteen mainline scaffolds could be connected when separated by 20 degrees.

Each arrangement of branches and limbs should be housed in a biocompatible, roughly cylindrical container 54. An entry and exit of approximately 1 to 2 cm diameter and 2 to 4 cm length exists at either end of the cylindrical unit.

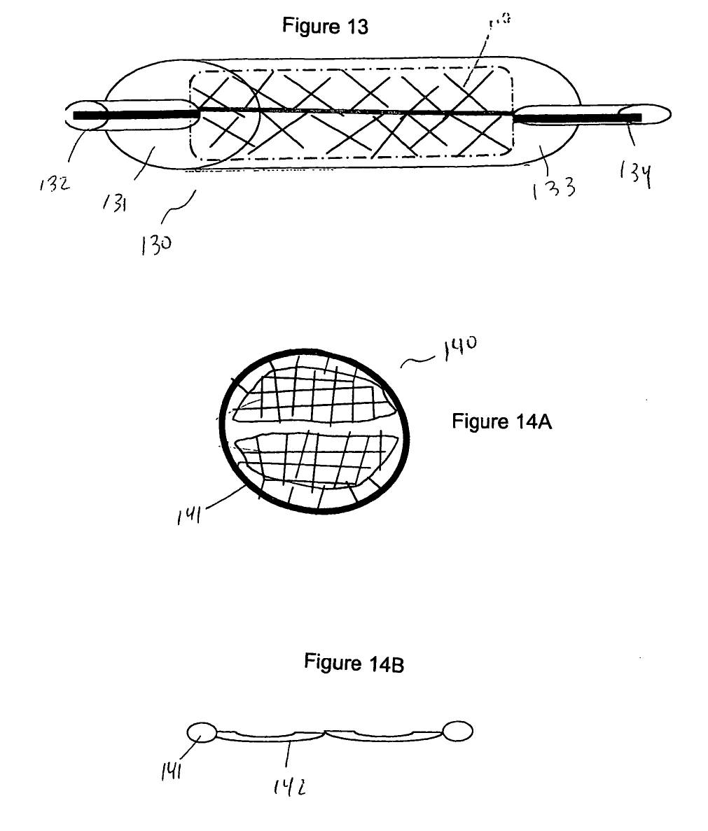

After preparing artificial pancreas 111, cells must be grown on the device. One way to accomplish the cell growth is through use of a pancreatic cell growth chamber 130 as depicted in FIG. 13. An entry region 131 includes an entry tube 132 to introduce a flow of cells. An exit region 133 collects additional fluid and/or cells and removes them through an exit tube 134. One artificial pancreas unit 100 with side branches 102 is shown inside the pancreatic cell growth chamber 130. Optionally, pancreatic cell growth chamber 130 could be used after multiple artificial pancreas units 100 had been configured into the desired shape and size.

Both the internal and external surfaces of each artificial pancreas unit 100 are coated with hormone producing islet cells. Preferably, donated islet cells (for example, from a pig, sheep, or goat) are separated into individual cells, digestive or enzymatic cells and islet or hormone cells. The islet or hormone cells are adhered to artificial pancreas unit 100 in, for example, a pancreatic cell growth chamber 130. If using human pancreas cells, matching donor and recipient blood antigens (A+, A−, B+, etc.) should be used to reduce the chance of rejection or complications.

Once complete, artificial pancreas 111 can be placed in an extremity of a patient. For example, in humans it is preferably placed in the forearm or thigh and sutured to arterial and venous vessels to allow for ease of insertion and use of local anesthetic. Arterial inflow is by way of primary anastomosis and venous outflow is via final anastomosis.

### EXAMPLE VI

### Synthetic Cardiac Valves

A top view of an artificial cardiac valve 140 is shown in FIG. 14A. Artificial cardiac valve 140 is composed of a circular valve ring 141 serving as an anchor and a plurality of leaflets 142. Attached to circular ring 141 and extending toward the center are preferably two or three leaflets 142, forming a two leaflet (bicuspid) or three leaflet (tricuspid) valve, respectively. Leaflets 142 are also composed of scaffold material, such as that of Example I. A side view of the valve is shown in FIG. 14B.

The valve diameter is approximately 2 to 4 cm. Once constructed, valve 140 will open in a first direction by flow forcing leaflets 142 to move in a uniform direction, opening the center of valve 140, defined by circular valve ring 141, for liquid to pass through. Valve 140 will close with opposite direction of flow. The opening and closing will be controlled by flow pressure.

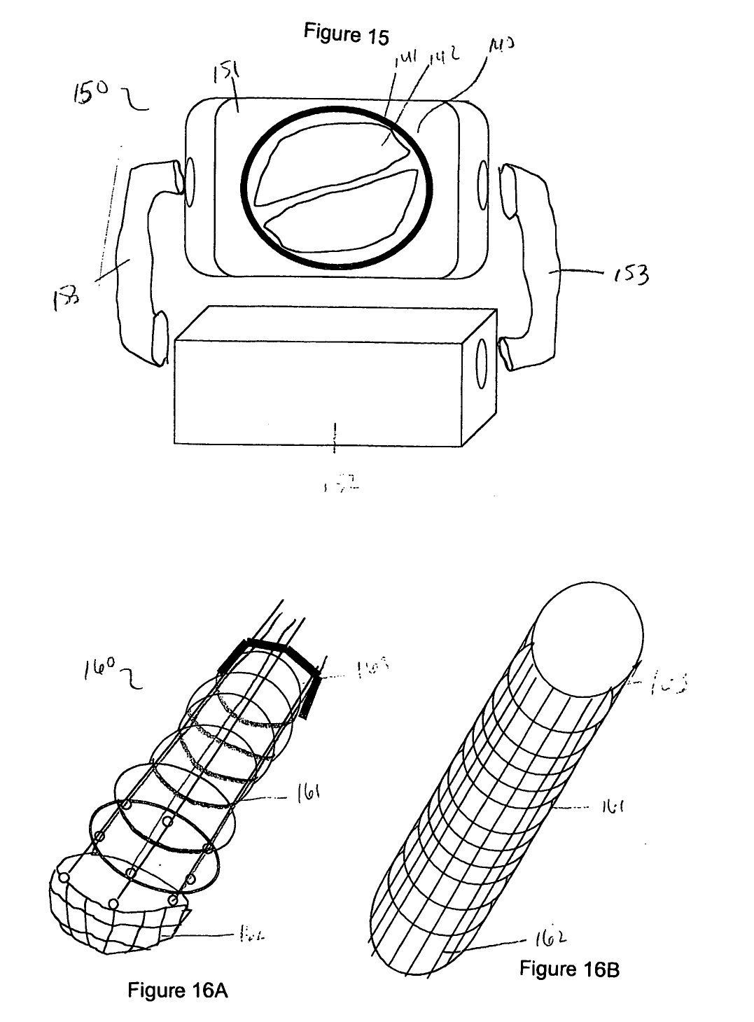

In order to provide the leaflet scaffolds with a cellular coating necessary for proper function, valve 140 can be placed in a cardiac valve cell growth chamber 150 prior to implant in a patient. Cardiac valve cell growth chamber 150 is shown in FIG. 15. After placing valve 140 in a treatment region 151 of cardiac valve cell growth chamber 150, a solution containing fibroblasts and/or cartilage cells is pumped over and around valve 140. The pumping is done by a pump and fluid reservoir 152. The cell culture solution travels from pump and fluid reservoir 152 to treatment region 151 through tubes 153. Two tubes 153 are shown, using two tubes 153 may be preferred to simulate the direction of blood flow valve 140 will eventually experience.

Optionally, endothelial cells may also be grown on valve 140 after some growth of fibroblasts and/or cartilage cells. Once seeded with cells, circular valve ring 141 could be sutured to native tissue or to a synthetic cardiac device.

Circular valve ring 141 can be constructed from any appropriate material, such as SILASTIC™, proline, methacrylate, nylon, dacron, polydimethylsiloxane, polyurethane, polyethylene, vicryl, gore-tex, polyproplene, octyl-2-cyanoacrylate, polymethylacrylate, polylactide, aramid, polystyrene, poly-L-lysine, latex, rubber, elastic, glass, ceramic, plastic, aluminum, copper, stainless steel, and titanium. The scaffold portion of valve 140 can be constructed with material such as SILASTIC™, proline, methacrylate, nylon, dacron, polydimethylsiloxane, polyurethane, polyethylene, vicryl, gore-tex, polyproplene, octyl-2-cyanoacrylate, polymethylacrylate, polylactide, aramid, polystyrene, poly-L-lysine, aluminum, copper, stainless steel, and titanium or other similar materials. Tubes 153 can be comprised of any desired material, the most appropriate materials may be those to which cells will not adhere.

### EXAMPLE VII

### Artificial Cardiac Ventricle

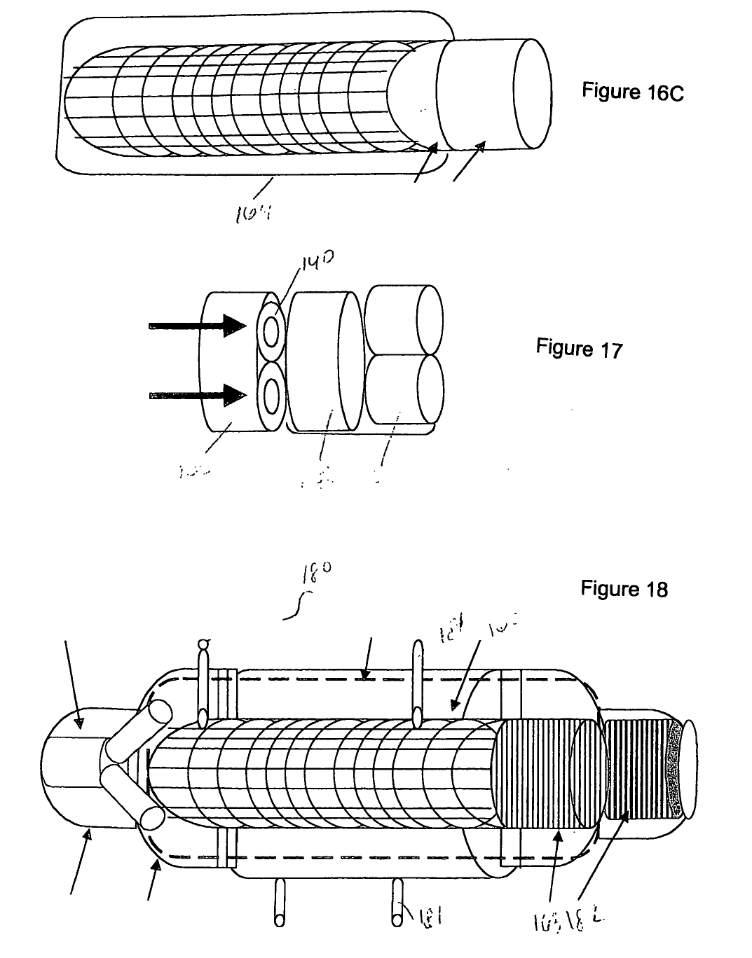

A further use for an artificial scaffold, such as that described in Example I, is for an artificial cardiac ventricle 160, depicted in FIG. 16A. Artificial cardiac ventricle 160 is comprised of a cylindrical scaffold 161 and is connected to a hemispheric base 162. Cylindrical scaffold 161 is wider at the end nearer base 162 than at the cylindrical scaffold apex 163. Apex 163 is open and may be attached to a valve, such as that described in Example VI. Grafted donor valves or artificial valves of any type may be used as desired. FIG. 16B shows a slightly less detailed view of artificial cardiac ventricle 160. The apex of the ventricle is attached to two valves, each unidirectional and opposite to one another. One valve allows fluid to flow into the ventricle, and the other allows fluid to flow out of the ventricle. While the two valves join on the inside of the ventricle to a single chamber, they remain separate outside the ventricle and are each joined to a distinct vessel or cardiac structure.

As depicted in FIG. 16C, artificial cardiac ventricle 160 is encased in a ventricle jacket 164. Ventricle jacket 164 protects artificial cardiac ventricle 160 from potential damage caused by materials and mechanics used to create a pumping action. FIG. 17 shows an attachment region for artificial cardiac ventricle 160, comprising a valve region 165. Valve region 165 is depicted with two valves and would be attached to an artificial cardiac ventricle (not shown). Valve region 165 is approximately 2.5 cm in diameter and 0.5 cm long. An optional dual valve extender 166 and two optional single valve extender 167 units are also shown. Extenders 166, 167 are approximately 2 to 3 cm long and can optionally be used to lengthen the attachment area that is utilized to connect valve region 165 to tubing that leads to a patient, or to a patient directly.

The valve region is made from prosthetic material, for example, dacron, gore-tex, polyethylene or polyurethane. Cardiac valves, either natural or artificial, such as those according to the present invention, are attached to the valve region with an appropriate material. The valve extender material may be from the same commercially available material as the valve region, or other material such as scaffold material of the present invention with an impermeable cell coating or an external coat of material sealant such as dacron, silastic, polyethylene, polyurethane or gore-tex. The valve, optional extender, and ventricle are attached in suture-like form with fibers or with a woven technique. One other option is to attach the valve and extender to the ventricle and to each other with adhesive material. Many materials noted through this disclosure would be appropriate for the attachments, for example, proline, nylon, stainless steel and methacrylate resin.

After artificial cardiac ventricle 160 is fabricated and placed in ventricle jacket 164, a cellular coating must be grown on the device. One means for growing cells on such a device is through the use of a cardiac ventricle cell growth chamber 180, shown in FIG. 18. Artificial cardiac ventricle 160 is placed inside cardiac ventricle cell growth chamber 180 and a cell culture solution containing cardiac muscle cells is introduced into cardiac ventricle cell growth chamber 180 through side ports 181. This allows a coating of cardiac muscle cells to adhere to artificial cardiac ventricle 160.

Cardiac ventricle cell growth chamber 180 also comprises an entry region 182 that connects to apex 163 of artificial cardiac ventricle 160. Through entry region 182, a cell culture solution of endothelial cells is passed around the inner side of artificial cardiac ventricle 160 and the endothelial cells are allowed to coat the scaffold interior.

Materials suitable for the scaffold of the artificial cardiac ventricle include SILASTIC™, proline, methacrylate, nylon, dacron, polydimethylsiloxane, polyurethane, polyethylene, vicryl, gore-tex, polyproplene, octyl-2-cyanoacrylate, polymethylacrylate, polylactide, aramid, polystyrene, poly-L-lysine, aluminum, copper, stainless steel, and titanium. Materials appropriate for the jacket include, inter alia, latex, rubber, elastic, ceramic and plastic. These materials are flexible and can withstand the effects of pump action and fluid pressure, both of which cause compression.

### EXAMPLE VIII

### Single Roller Wheel Pump

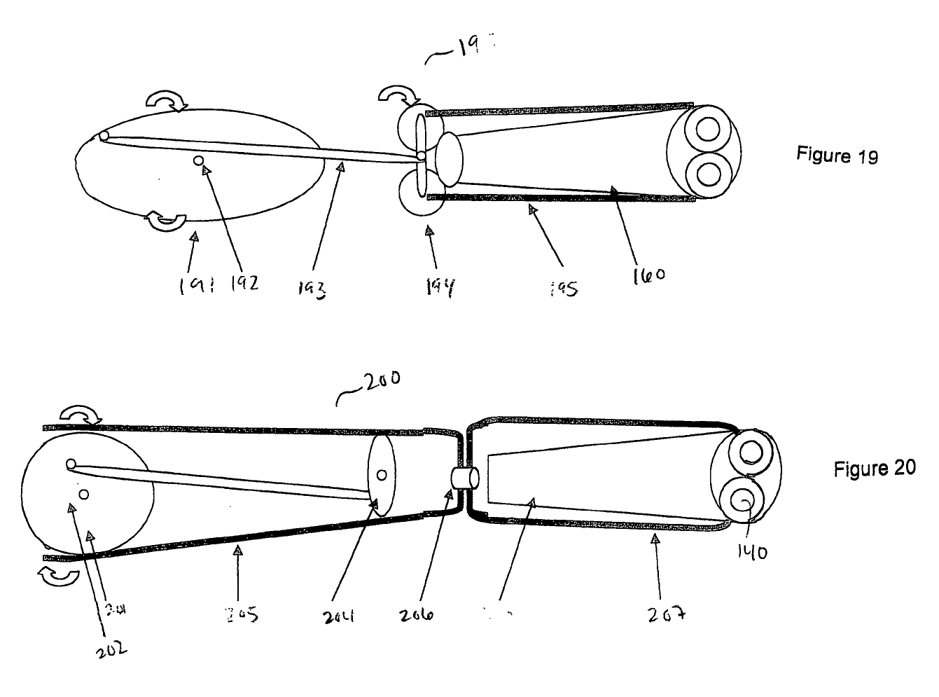

As depicted in FIG. 19, a pump 190 is provided to move fluid into and out of an artificial cardiac ventricle such as that described in Example VII. Pump 190 connects to a drive (not shown) that spins a crank wheel or drive wheel 191 around a drive wheel axis 192. Motion of drive wheel 191 causes a drive rod 193 to reciprocate. Drive rod 193 moves a connecting bar 196 which in turn moves a pair of ventricle compression wheels 194. Ventricle compression wheels 194 travel along wheel guide rails 195 and cause rhythmic compression and decompression of artificial cardiac ventricle 160. This rhythmic compression simulates normal cardiac pumping in an organism. Specifications such as timing and pressure can be regulated according to the needs of the organism. A variable speed drive can be provided for adjustable pump attributes.

Wheels 194 are preferably constructed to be firm enough to cause compression yet somewhat yielding and smooth. Wheels 194 are preferably coated with a material that is similar or identical to the material coating artificial cardiac ventricle 160. Guide rails 195 may be coated with a lubricant, for example, a silicone based oil. The lubricant need not be in contact with artificial cardiac ventricle 160, as guide rails 159 can be shielded or contained within a housing.

### EXAMPLE IX

### Liquid Force Pump

An alternative means to achieve rhythmic compression of artificial cardiac ventricle 160 and simulate normal cardiac pumping is through the use of a liquid force pump 200, depicted in FIG. 20. Liquid force pump 200 operates through the use of increasing and decreasing fluid pressure instead of manual compression as described with the pump of Example VIII.

Liquid force pump 200 is powered by a motor (not shown) that connects to a drive wheel axis 201 and rotates a drive wheel 202. Drive wheel 202 is connected to a drive rod 203 and causes drive rod 203 to reciprocate back and forth as drive wheel 202 rotates. The motion of drive rod 203 causes a corresponding motion in plunger 204. Liquid force pump 200 is contained within a pump container 205 having an exit port 206. Plunger 204 forms a watertight seal inside pump container 205. Liquid is contained in the region between plunger 204 and exit port 206, the back and forth motion of plunger 205 forces liquid within pump container 205 to be forced out or drawn into pump container 205.

One other component of this pumping system is a cardiac device container 207 in fluid communication with pump container 205 and through port 206. Cardiac device container 207 houses artificial cardiac ventricle 160 and contains liquid. As liquid force pump 200 causes liquid to be expelled from pump container 205 through exit port 206, liquid enters cardiac device container 207. Fluid volumes and container 205, 207 sizes are adjusted so that pressure within cardiac device container 207 increases enough to expel liquid from inside artificial cardiac ventricle through valve 140. As plunger 204 moves back toward drive wheel 202, liquid is drawn into pump compartment 205 and removed from cardiac compartment 207. This movement of liquid causes a corresponding drop in pressure inside artificial cardiac ventricle 160 and fluid is drawn into artificial cardiac ventricle 160.

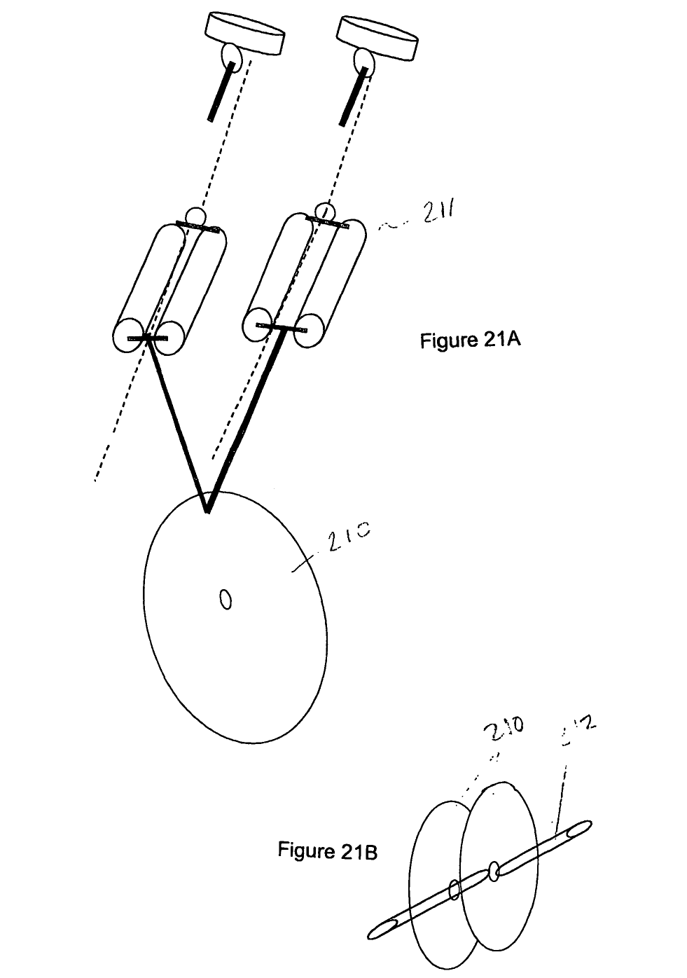

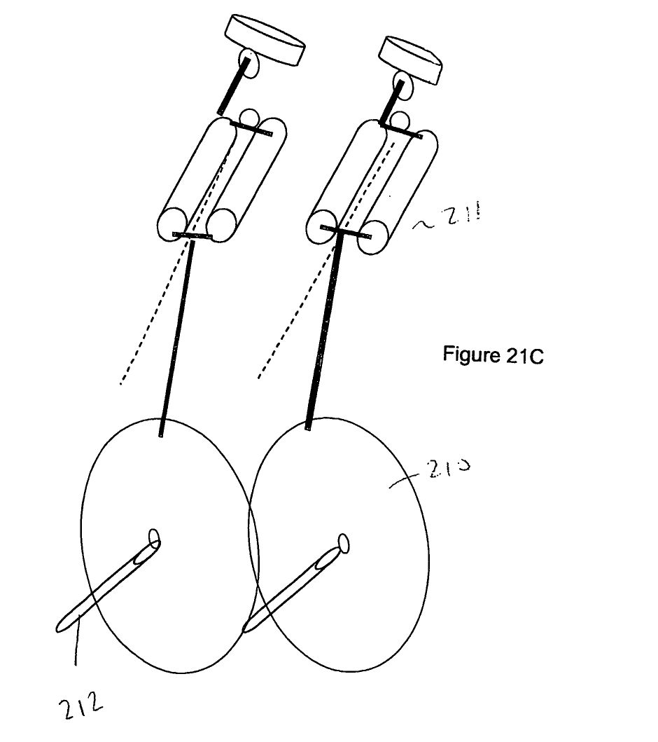

Additional components of pumps according to Examples VIII and IX are shown in FIGS. 21 A, 21B, and 21C. FIG. 21A shows a single drive wheel 210 connected to two pumping regions 211. A pump configured accordingly would be capable of compressing two artificial cardiac ventricles at the same time and with the same rhythmic compression. FIG. 21B shows a single drive wheel axis 212 connected to two drive wheels 210. FIG. 21C shows another optional arrangement of two drive wheels 210 adjacent to one another, with individual drive wheel axis 212. Each drive wheel 210 is connected to a single pumping region 211. By connecting two drive rods to a drive mechanism 180° out of phase, an alternating pumping action can be achieved.

### EXAMPLE X

### Internal Cardiac Device

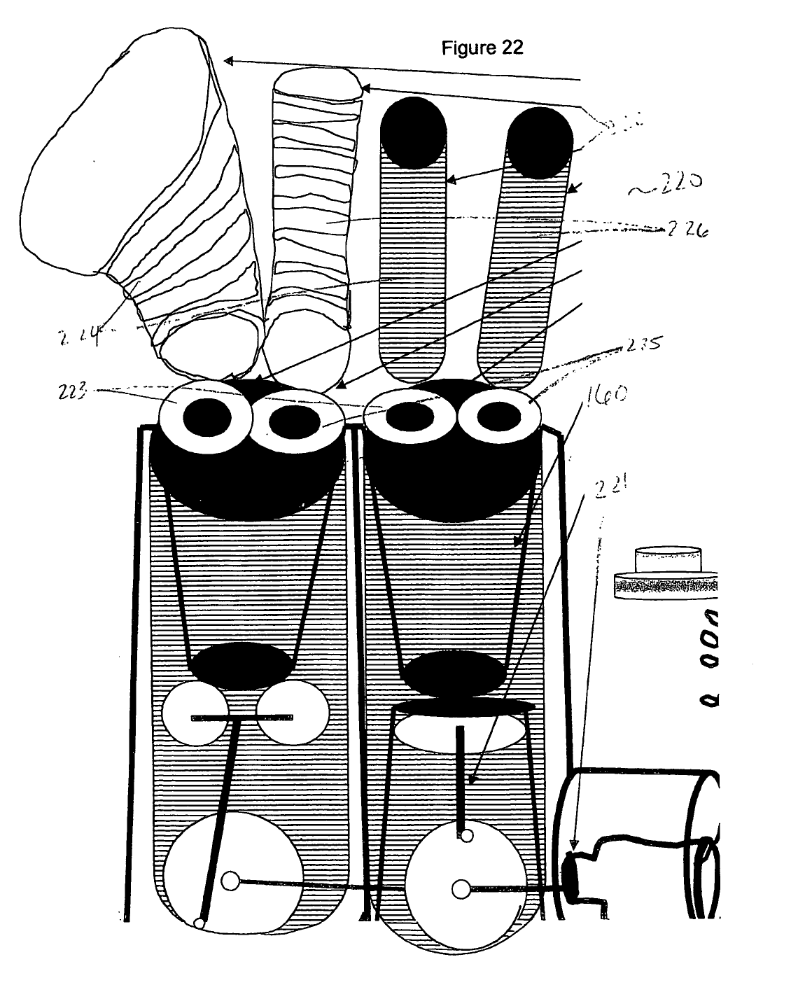

FIG. 22 depicts an internal cardiac device 220. Internal cardiac device 220 is comprised of a pair of artificial cardiac ventricles 160, a pumping means 221 to create rhythmic compression of artificial cardiac ventricles 160, and connection means 222 establishing fluid communication between artificial cardiac ventricles 160 and a patient (not shown).

Pumping means 221 creates compression and decompression by any means determined appropriate, for example, according to either Example VIII or IX, above. When artificial cardiac ventricles 160 are compressed, liquid within artificial cardiac ventricles 160 is forced out an exit valve 223 through exit arteries 224 and into the patient (not shown). Following compression, pumping means 221 causes decompression of artificial cardiac ventricles 160, causing an intake of fluid through intake valves 225 from intake veins 226. Intake veins connect to a patient's vein (not shown).

The motor or other equipment used to propel pumping means 221, including any power source, may be located inside or outside a patient. The most appropriate source, size, and location can be determined by those skilled in the art, within the scope of the present invention. For example, a variable speed pumping means may be powered by a battery located subcutaneously on a patient. An external device could be held in proximity to the subcutaneous battery and inductively recharge the battery.

### EXAMPLE XI

### External Cardiac Device

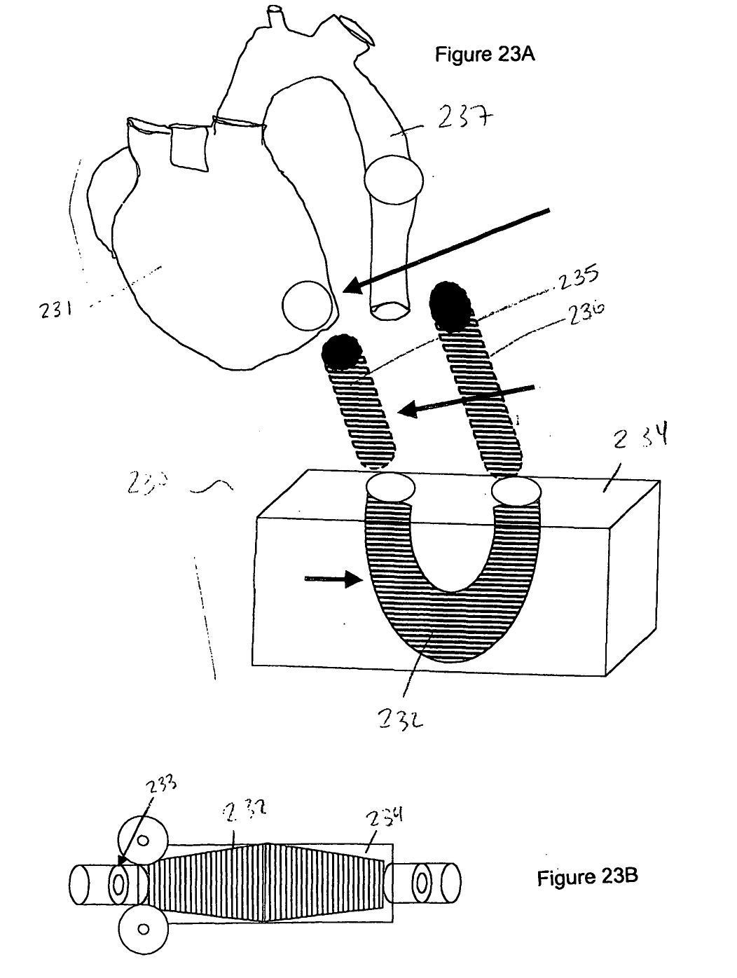

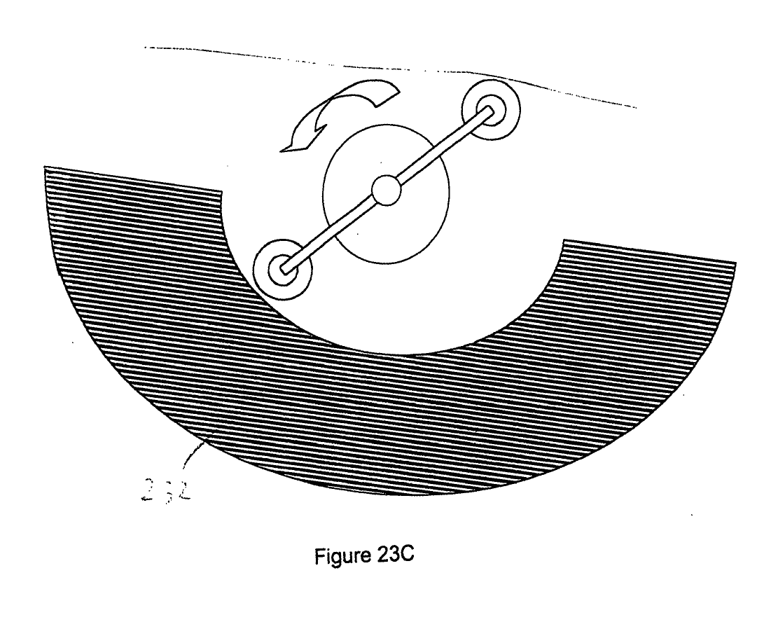

As depicted in FIG. 23A, an external cardiac device 230 can be employed when one portion of a patient's heart 231 is not functioning properly. External cardiac device 230 comprises a cylindrical artificial cardiac device 232 similar to that described in Example VII, except that artificial cardiac device 232 is open at both ends. Artificial cardiac device 232 can be formed from a cell-coated scaffold as described in previous Examples. A valve 233, whether artificial or donor, is located at each end of artificial cardiac device 232.

Artificial cardiac device 232 is housed in a pumping region 234. Pumping region 234 has a pumping means that forces fluid through artificial cardiac device 232. FIG. 23B shows one configuration of pumping region 234, with a pair of wheels provided to roll along the length of artificial cardiac device 232 and force fluid through. FIG. 23C shows yet another embodiment of the pumping means, a roller wheel that rotates and causes compression along one side of artificial cardiac device 232. Pumping means may include a reservoir (not shown) to supply the region between artificial cardiac device 232 and the outer encasement of pumping region 234 with a sterile fluid such as plasma.

Regardless of pumping means ultimately selected, external cardiac device 230 is connected to patient's heart 231 through a first connecting tube 235 and a second connecting tube 236. First connecting tube 235 is connected at a first end to patient's heart 231 in the region of defect, for example, the right ventricle. First connecting tube 235 connects at a second end to one valve 233 of artificial cardiac device 232. Second connecting tube 236 connects at a first end to another valve 233 of artificial cardiac device 232 and at a second end to a patient's artery 237, for example, the aorta. External cardiac device 230 can be fabricated of materials described above or other appropriate materials known in the art. External cardiac device 230 may be used temporarily in a patient or on a permanent basis as determined by the practitioner. Connecting tubes 235, 236 could be fabricated from an impermeable material, such as heparin bounded dacron, polyethylene, polyurethane or goretex with heparin or dextran bounded pharmaceutical and a flexible stabilizing sealant or outer container.

### EXAMPLE XII

### Formation of Artificial Artery, Using Porous Polymer Tubing as Scaffold

A porous polymeric material was fabricated as a scaffold upon which smooth muscle and endothelial cells were grown, to yield a durable, elastic and non-thrombogenic device suitable as an artery replacement or graft. The surgical placement of similar grafts is known in the art.

Microporous flexible tubing was fabricated. Novel microporous soft tubes were made of nylon-11, —[—(CH₂)₁₀CONH]— (MW approximately 200,000). The tubes had approximately 70% porosity, 2 micron pore size (as determined by scanning electron microscopy (SEM), were non-swelling in water, lacked both additives and plasticizers, and showed good strength and durability. Tubes of various diameters were made by extruding a formulation of nylon-11 and a water-soluble polyethylene oxide. Polyethylene oxide (PEO) polymers have the common structural moiety of —(OCH₂CH₂)ₙ—OH. One embodiment of the instant invention for an artificial blood vessel is given in the following table:

TABLE A

| Ingredient | Nylon-11, wt %¹ | PolyOx² | CaCO₃ | Total |
|---|---|---|---|---|
| Weight % | 0.185 | 0.457 | 0.358 | 1.00 |
| Volume % | 0.255 | 0.57 | 0.175 | 1.00 |
| Weight (g) | 148 | 365.6 | 286.4 | 800 |

¹Nylon-11 available from Elf Atochem
²PolyOx available from Union Carbide

After extrusion, the tubes were soaked in water. The polyethylene oxide dissolved in water and was removed from the tube. The pores in the nylon tubing were thus generated as a result of the water extraction of the polyethylene oxide. Pore size and porosity of the nylon tubing was varied, based on the formulation as well as the extrusion conditions. Good control of porosity over a range of approximately 50 to 80% porosity was obtained. Pore size was controlled within a range between approximately 0.5 micron to 5 micron.

Nylon-11 is useful as a scaffold material for bioartificial tissue fabrication since it is water-insoluble, has no additives/plasticizers, and has excellent durability. In contrast, the more commonly used nylon-6 or nylon-6,6 can swell in water, and their overall thermomechanical resiliency is less desirable over longer times in comparison with nylon-11.

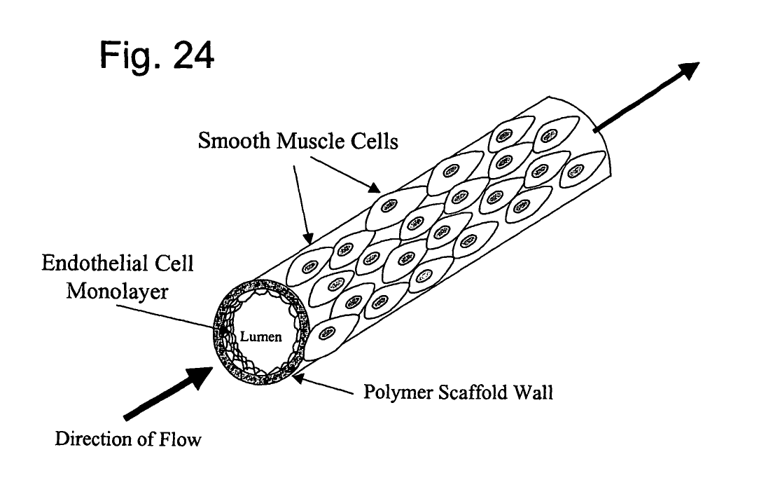

A bioreactor was used to selectively grow smooth muscle cells and endothelial cells on the porous tube to produce a functional artery. FIG. 24 is a schematic illustration of a resulting synthetic artery. The polymer scaffold wall is coated on the exterior by a uniform layer of vascular smooth muscle cells and on the interior by a monolayer of vascular endothelial cells. The arrows show the direction of flow of the cell culture solutions. The exterior of the scaffold tube was contacted with a cell culture solution of vasclular smooth muscle cells and the interior with a cell culture solution of vascular endothelial cells. Combining grown natural cells with a flexible, tough and highly porous polymer tube leads to a synthetic device that can be used as an artificial organ replacement for, for example, arteries.

The porous tube was sanitized, then placed in a cartridge with two chambers separated by the porous polymer tube scaffold. The outer chamber is in direct contact with the outer wall of the polymer tube and was in contact with a solution containing vascular smooth muscle cells (VSMC's). The inner chamber (inside the porous scaffold) was filled with a solution containing endothelial cells. The tube was incubated for two days, after which time longitudinal flow was applied to the inner chamber. After two weeks, the tube was removed. The outer surface of the scaffold tube contained a layer of VSMCs, and the inner surface of the scaffold tube was lines with endothelial cells. The endothelial cells were properly aligned.

In this device, the matrix polymer remained intact, and provided strength to the device, in contrast to other devices which rely on dissolution of a matrix. The specific morphology and chemical nature of the polymer scaffold results in a uniform cell coverage, in a timeframe much shorter than previously obtained with multi-filament polymer designs or scaffolds designed to be dissolved over time in a host.

The resulting synthetic vessel may be implanted by known techniques heretofore used to implant analogous devices.

### EXAMPLE XIII

### Controlling Scaffold Porosity by Solvent Choice

The use of a (water) soluble co-extrudable thermoplastic (e.g., polyethylene oxide, POLYOX® Water Soluble Resins, nonionic water-soluble poly(ethylene oxide) polymers with the common structure: —(OCH₂CH₂)ₙ—OH) with an insoluble thermoplastic (e.g., nylon or TPU) to obtain control over porosity can be extended. Alternatively, following the procedure as detailed in Example XII, alcohol is used instead of water for the polyethylene oxide extraction. Changing the extraction solvent, either alone or as a co-solvent, including variations of pH, will enable the skilled artisan to control porosity via selective solubilities. Such fine tuning will lead to higher degrees of morphological resolution of the scaffold and resulting material. Examples of water-miscible solvents include solvents such as anhydrous isopropyl alchohol, ethylene glycol, propylene glycol, anhydrous ethanol, glycerin, Cellosolve, Carbitol, and/or inorganic salt solutions.

The use of other types of solvents and/or solvent systems makes it possible to broaden the variety of polymer materials which can be used in the invention. The Hildebrand solubility parameters known for each of the polymeric materials would be a useful criteria for optimizing the greatest degree of differential solubility. This higher selectivity in solvent types leads to a higher degree of control over the porosity of the final scaffold.

It is also possible to extend control over the porosity of the resulting scaffold matrix by incorporating a soluble filler in the polymer mixture. For example, inorganic fillers, with lower water solubility, are used to seed a matrix material in order to increase strength and decrease flexibility. Such scaffold materials are used to form artificial bone and/or cartilage. For example, calcium carbonate, CaCO₃, which is partially water soluble, can be incorporated in the polymer material fed to the extrusion or molding step and then dissolved away to adjust the porosity of the scaffold.

Although nylon-11 may not be appropriate for all arterial targets (i.e., it may be too rigid), it is useful as a core scaffold for the manufacture of an artificial liver. For more flexible and rubber-like porous materials, part or all of the nylon is substituted with a thermoplastic urethane (TPU).

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

### REFERENCES

References Discussing Various Materials Useful in the Practice of the Present Invention. e.g. Silicone, Proline, Plastic, Polystyrene, Polypropylene, Polygalactin, Polylactide, Glass, Aramid, and Dacron:

1: Slavicek A, Betka J, Novak V, Kinkal J, Wasserbauer R, Vymazalova Z, Last'ovickova B. Sb Lek. 2000;101(4):325-39. Czech. PMID: 11702571 [PubMed—in process]
2: Goodman G J. Management of post-acne scarring. What are the options for treatment? Am J Clin Dermatol. January-February 2000;1(1):3-17. PMID: 11702303 [PubMed—in process]
3: Brandon H J, Young V L, Jerina K L, Wolf C J. Related Articles Variability in the properties of silicone gel breast implants. Plast Reconstr Surg. September 2001; 108(3):647-5 5. PMID: 11698836 [PubMed—in process]
4: von Der Thusen J H, Kuiper J, Fekkes M L, de Vos P, van Berkel T J, Biessen E A. Related Articles Attenuation of atherogenesis by systemic and local adenovirus-mediated gene transfer of interleukin-10 in LDLr −/−Mice. FASEB J. Oct. 29, 2001 [epub ahead of print] PMID: 11687507 [PubMed—as supplied by publisher]
5: Samarakkody U, Klaassen M, Nye B. Related Articles Reconstruction of congenital agenesis of hemidiaphragm by combined reverse latissimus dorsi and serratus anterior muscle flaps. J Pediatr Surg. November 2001;36(11):1637-40. PMID: 11685690 [PubMed—in process]
6: Hiraki Y, Shukunami C, lyama K, Mizuta H. Related Articles Differentiation of chondrogenic precursor cells during the regeneration of articular cartilage. Osteoarthritis Cartilage. 2001;9 Suppl A: S102-8. PMID: 11680673 [PubMed—in process]
7: Ricci B, Ricci F. Related Articles Octyl 2-cyanoacrylate tissue adhesive in experimental scleral buckling. Acta Ophthalmol Scand. October 2001;79(5):506-8. PMID: 11594989 [PubMed—in process]
8: Doherty C. Related Articles Vertical banded gastroplasty. Surg Clin North Am. October 2001;81(5):1097-112. Review. PMID: 11589247 [PubMed—indexed for MEDLINE]
9: Herz D, Hafez A, Bagli D, Capolicchio G, McLorie G, Khoury A. Related Articles Efficacy of endoscopic subureteral polydimethylsiloxane injection for treatment of vesicoureteral reflux in children: a North American clinical report. J Urol. November 2001; 166(5):1880-6. PMID: 11586254 [PubMed—indexed for MEDLINE]
10: Snyder C L, Miller K A, Sharp R J, Murphy J P, Andrews W A, Holcomb G W 3rd, Gittes G K, Ashcraft K W. Related Articles Management of intestinal atresia in patients with gastroschisis. J Pediatr Surg. October 2001;36(10):1542-5. PMID: 11584405 [PubMed—in process]
11: Gori T, Mak S S, Kelly S, Parker J D. Related Articles Evidence supporting abnormalities in nitric oxide synthase function induced by nitroglycerin in humans. J Am Coll Cardiol. October 2001;38(4):1096-101. PMID: 11583888 [PubMed—indexed for MEDLINE]
12: Xiang D Z, Verbeken E K, Van Lommel A T, Stas M, De Wever 1. Related Articles Sleeve-related thrombosis. a new form of catheter-related thrombosis. Thromb Res. Oct. 2, 2001;104(1):7-14. PMID: 11583734 [PubMed—in process]
13: Wungcharoen B, Pradidarcheep W, Santidhananon Y, Chongchet V. Related Articles Pre-arterialisation of the arterialised venous flap: an experimentalstudy in the rat. Br J Plast Surg. October 2001;54(7):621-30. PMID: 11583500 [PubMed—in process]
14: Chabinyc M L, Chiu D T, McDonald J C, Stroock A D, Christian J F, Karger A M, Whitesides G M. Related Articles An integrated fluorescence detection system in poly(dimethylsiloxane) for microfluidic applications. Anal Chem. Sep. 15, 2001;73(18):4491-8. PMID: 11575798 [PubMed—indexed for MEDLINE]
15: Rajkumar L, Guzman R C, Yang J, Thordarson G, Talamantes F, Nandi S. Free in PMC, Related Articles Short-term exposure to pregnancy levels of estrogen prevents mammary carcinogenesis. Proc Natl Acad Sci U S A. Sep. 25, 2001;98(20): 11755-9. PMID: 11573010 [PubMed—in process]
16: Biffi R, De Braud F, Orsi F, Pozzi S, Arnaldi P, Goldhirsch A, Rotmensz N, Robertson C, Bellomi M, Andreoni B. Related Articles A randomized, prospective trial of central venous ports connected to standard open-ended or Groshong catheters in adult oncology patients. Cancer. Sep. 1, 2001;92(5):1204-12, PMID: 11571734 [PubMed—indexed for MEDLTNE]
17: Mottura A A. Related Articles Short columella nasolabial complex in aesthetic rhinoplasty. Aesthetic Plast Surg. July-August 2001;25(4):266-72. PMID: 11568829 [PubMed—indexed for MEDLINE]
18: Atkins K B, Johns D, Watts S, Clinton Webb R, Brosius III F C. Related Articles Decreased vascular glucose transporter expression and glucose uptake in DOCA-salt hypertension. J Hypertens. September 2001; 19(9): 1581-7. PMID: 11564977 [PubMed—in process]
19: Zook B C, Janne O A, Abraham A A, Nash H A. Related Articles The development and regression of deciduosarcomas and other lesions caused by estrogens and progestins in rabbits. Toxicol Pathol. July-August 2001;29(4):411-6. PMID: 11560245 [PubMed—in process]
20: Spector B C, Netterville J L, Billante C, Clary J, Reinisch L, Smith T L. Related Articles Quality-of-life assessment in patients with unilateral vocal cord paralysis. Otolaryngol Head Neck Surg. September 2001;125(3): 176-82. PMID: 11555751 [PubMed—indexed for MEDLINE]
   References Discussing Nylon Materials Useful in the Practice of the Present Invention:
21: Siragusa M, Ferri R, Cavallari V, Schepis C. Friction melanosis, friction amyloidosis, macular amyloidosis, towel melanosis: many names for the same clinical entity. Eur J Dermatol. November-December 2001; 11(6):545-8.
22: Fontaine L, Eveii S, Soucaille P, Lindley N D, Cocaign-Bousquet M. Transcript quantification based on chemical labeling of RNA associated with fluorescent detection. Anal Biochem. Nov. 15, 2001;298(2):246-52.
23: Rabelo E, Bertics S J, Mackovic J. Grummer R R. Strategies for increasing energy density of dry cow diets. J Dairy Sci. October 2001;84(10):2240-9.
24: Mayne M, Cheadle C, Soldan S S, Cermelli C, Yamano Y, Akhyani N, Nagel J E, Taub D D, Becker K G, Jacobson S. Gene expression profile of herpesvirus-infected T cells obtained using immunomicroarrays: induction of proinflammatory mechanisms. J Virol. December 2001;75(23):11641-50.
25: Lappin S, Cahlik J, Gold B. Robot printing of reverse dot blot arrays for human mutation detection. J Mol Diagn. November 2001;3(4):178-88.
26: Joussen A M, Huaiig S. Poulaki V, Camphausen K, Beecken W D, Kirchhof B Adamis A P. In Vivo Retinal Gene Expression in Early Diabetes. Invest Ophthalmol Vis Sci. November 2001;42(12):3047-3057.
27: Tognetto D, Agolini G, Grandi G, Ravalico G. Iris alteration using mechanical iris retractors. J Cataract Refract Surg. October 2001;27(10):1703-5.
28: O'Brien P A, Marfleet C. Frameless versus classical intrauterine device for contraception (cochrane review). Cochrane Database Syst Rev. 2001;4:CDO03282.
29: Gunzer M, Weishaupt C, Planelles L, Grabbe S. Two-step negative enrichment of CD4(+) and CD8(+) T cells from murine spleen via nylon wool adherence and an optimized antibody cocktail. J Immunol Methods. Dec. 1, 2001;258(1-2):55-63.
30: Zana K, Otal P. Rousseau H, Joffre F. Fornet B. Orv Hetil. Aug. 26, 2001; 142(34):1837-41. Hungarian.
31: Dupre 1, Atifi M E, Rostaing B, Chambaz E M, Benabid A L, Berger F. Microarray RNA quantification assay for large-scale nanosamples analysis. Biotechniques. October 2001;31(4):856-8, 860.
32: Pividori M L Merkoci A, Aleuret S. Classical dot-blot format implemented as an amperometric hybridisation genosensor. Biosens Bioelectron. December 2001; 16(9-12):1133-42. PMID: 11679299
33: Millan F R, Lovez Pla S, Roa Tavera V, Tapia M S, Cava R. Arch Latinoam Nutr. June 2001;51(2):173-9. Spanish.
34: John J. Gangadhar S A, Shah I. Flexural strength of heat-polymerized polymethyl methacrylate denture resin reinforced with glass, aramid, or nylon fibers. J Prosthet Dent. October 2001;86(4):424-7. PMID: 11677538 [PubMed—in process]
35: Lju Y, Belgyev L, Zhao W, Busto R, Belayev A, Ginsberg M D. Neuroprotective effect of treatment with human albumin in permanent focal cerebral ischemia: histopathology and cortical perfusion studies. Eur J Pharmacol. Oct. 5, 2001;428(2):193-201.
36: Lumbikanonda N, Sammons R. Bone cell attachment to dental implants of different surface characteristics. Int J Oral Maxillofac Implants. September-October 2001; 16(5):627-36.
37: Alattar M H, Ravindranath T M, Choudhry M A, Muraskas J K, Narnak S Y, Dallal O, Sayeed M M. Sepsis-induced alteration in t-cell ca(2+) signaling in neonatal rats. Biol Neonate. October 2001;80(4):300-4.
38: Quan J, Du G. [Protective effect of Astragalus membranaceus (Fisch.) Bge. and Hedysarum polybotrys Hand.-Mazz. on experimental model of cerebral ischernia in rats]. Zhongguo Zhong Yao Za Zhi. June 1998;23(6):371-3, inside back cover. Chinese.
39: Manoonkitiwongsa P S, Jackson-Friedman C, McMillan P J, Schultz R L, Lyden P D. Angiogenesis after stroke is correlated with increased numbers of macrophages: the clean-up hypothesis. J Cereb Blood Flow Metab. October 2001;21(10):1223-31. Review.
40: Henderson L A, Fjysinger R C, Yu P L, Bandler R, Harper R M A device for feline head positioning and stabilization during magnetic resonance imaging. Magn Reson Imaging September 2001; 19(7):1031-6.
41: Eur J Dermatol November-December 2001; 11(6):545-8 Friction melanosis, friction amyloidosis, macular amyloidosis, towel melanosis: many names for the same clinical entity
42: Siragusa M, Ferri R, Cavallari V, Schepis C. Unit of Dermatology, Oasi Institute for Research on Mental Retardation and Brain Aging (IRCCS), Via Conte Ruggero, 73, 94018 Troina, Italy.
   References Discussing Proline Materials Useful in the Practice of the Present Invention:
43: el-Enany A E. Issa A A. Proline alleviates heavy metal stress in Scenedesmus armatus. Folia Microbiol (Praha). 2001;46(3):227-30.
44: Lam B C, Sne T L, Bianchi F, Blumwald E. Role of SH3 Domain-Containing Proteins in Clathrin-Mediated Vesicle Trafficking in Arabidopsis. Plant Cell. November 2001; 13(11):2499-512.
45: Paolini C L, Marconi A M, Ronzoni S, Di Noio M, Fennessey P V, Pardi G, Battagu F C. Placental transport of leucine, phenylalanine, glycine, and proline in intrauterine growth-restricted pregnancies. J Clin Endocrinol Metab. November 2001;86(11):5427-32.
46: Kallio J, Pesonen U, Karvonen M K, Kojima M, Hosoda H, Kan˜),awa K, Koulu M. Enhanced Exercise-Induced GH Secretion in Subjects with Pro7 Substitution in the Prepro-NPY. J Clin Endocrinol Metab. November 2001;86(11):5348-52.
47: Reiersen H, Rees A R. The hunchback and its neighbours: proline as an environmental modulator. Trends Biochem Sci. November 2001;26(11):679-84.
48: Hamase K, Inoue T, Morikawa A, Konno R, Zaitsu K. Determination of Free d-Proline and d-Leucine in the Brains of Mutant Mice Lacking d-Amino Acid Oxidase Activity. Anal Biochem. Nov. 15, 2001;298(2):253-8.
49: Oda T, Muramatsu Ma M A, Ism,,ai T, Masuho Y, Asano S, Yamashita T. HSH2: A Novel SH2 Domain-Containing Adapter Protein Involved in Tyrosine Kinase Signaling in Hematopoietic Cells. Biochem Biophys Res Commun. Nov. 16, 2001;288(5):1078-86.
50: Galoyan A A, Sarkissian J S, Kipriyan' F K, Sarkissian E J. Chavushvan F A, Sulkhanyan R M, Meliksetyan 113, Abrahamyan S S. Grigorian Y K h, Avetisyan Z A, Otieva N A. Protective effect of a new hypothalamic peptide against cobra venom and traumainduced neuronal injury. Neurochern Res. September 2001;26(8-9):1023-38.
51: Marshall D, Hardman M J, Nield K M, Byrne C. Differentially expressed late constituents of the epidermal cornified envelope. Proc Natl Acad Sci U S A. Nov. 6, 2001;98(23):13031-6.
52: Nakanishi T, Kekuda R, Fei Y J. Hatanaka T, Swaawara M, Martindale R G, Leibach F H, Prasad P D, Ganqpathy V. Cloning and functional characterization of a new subtype of the amino acid transport system N. Am J Physiol Cell Physiol. December 2001;281(6):C1757-68.
53: Karna E, Pal&z shtsls;ka J, Wol&z slitsls;czynski S. Doxycycline-induced inhibition of prolidase activity in human skin fibroblasts and its involvement in impaired collagen biosynthesis. Eur J Pharmacol. Oct. 26, 2001;430(1):25-31.
54: Wang C, Pawley N H, Nicholson L K. The Role of Backbone Motions in Ligand Binding to the c-Src SH3 Domain. J Mol Biol. Nov. 2, 2001;313(4):873-887
55: Lefevre F, Garnotel R, Georges N, Gillery P. Modulation of collagen metabolism by the nucleolar protein fibrillarin. Exp Cell Res. Nov. 15, 2001;271(1):84-93.
56: Sano K. Structure of AF3p2 I, a new member of mixed lineage leukemia (MLL) fusion partner proteins-implication for MLL-induced leukemogenesis. Leuk Lymphoma. August 2001;42(4):595-602.
57: Ilveskoski E, Kajander O A, Lehtimaki T, Kunnas T, Karhunen P J, Heinala P, Virkkunen M. Alho H. Association of neuropeptide y polymorphism with the occurrence of type I and type 2 alcoholism. Alcohol Clin Exp Res. October 2001;25(10): 1420-2.
58: Boras K, Hamel P A. Alx4 binding to LEF-I regulates N-CAM promoter activity. J Biol Chem. Nov. 5, 2001
59: Ye S Q, Chu C C, Cao S Y, Tang, Z S, Wang L, Zhao S M, Tian W Z. The factors of improving rice transformation efficiency]. Yi Chuan Xue Bao. 2001;28(10):933-8. Chinese.
60: Ringli C, Keller B, Ryser U. Glycine-rich proteins as structural components of plant cell walls. Cell Mol Life Sci. September 2001;58(10):1430-41.
61: Kieliszewski M J, Shpak E. Synthetic genes for the elucidation of glycosylation codes for arabinogalactanproteins and other hydroxyproline-rich glycoproteins. Cell Mol Life Sci. September 2001;58(10):1386-98.
62: Natesan S, Reddy S R. Compensatory changes in enzymes of arginine metabolism during renal hypertrophy in mice. Comp Biochem Physiol B Biochem Mol Biol. December 2001;130(4):585-95.
   References Discussing Various Plastic Surgery Methods and Materials Useful in the Practice of the Present Invention:
63: Alarcon T, Lopez-Hernandez S, Andreu D, Saugar J M, Rivas L, Lopez-Brea M. In vitro activity of CA(I-8)M(I-18), a synthetic cecropin A-melittin hybrid peptide, against multiresistant Acinetobacter baumannii strains. Rev Esp Quirnioter. June 2001; 14(2):184-90
64: Otto C, Baumann M, Schreiner T, Bartsch G, Borbery H, Schwandt P, SchmidSchonbein H. Standardized ultrasound as a new method to induce platelet aggregation. Evaluation, influence of lipoproteins and of glycoprotein IIb/IIIa antagonist tirofiban. Eur J Ultrasound. December 2001; 14(2-3):157-166.
65: Cartling B. Neuromodulatory control of interacting medial temporal lobe and neocortex in memory consolidation and working memory. Behav Brain Res. Nov. 29, 2001;126(1-2):65-80.
66: Gehrking E, Klostermann W, Wessel K, Remmert S. [Electromyography of the Infrahyoid Muscles—Part 1: Normal Findings]. Laryngorhinootologie. November 2001;80(11):662-665. German.
67: Engelke K, Suss C, Kalelider W A. Stereolithographic models simulating trabecular bone and their characterization by thin-slice- and micro-CT. Eur Radiol. October 2001; I 1(10):2026-2040.
68: Takaki M, Ujike H, Kodama M, Takehisa Y, Nakata K, Kuroda S. Two kinds of mitogen-activated protein kinase phosphatases, MKP-I and MKP-3, are differentially activated by acute and chronic methamphetamine treatment in the rat brain. J Neurochem. November 2001;79(3):679-88.
69: Luftl M, Schuler G, Kiesewetter F. Tinea and Erysipelas carcinomatosum. Eur J Dermatol. November-December 2001; 11(6):593-4.
70: de Bono S. Plastic protein mimics. Trends Biochem Sci. November 2001;26(11):647. No abstract available.
71: Dobbins I G. The systematic discrepancy between A′ for overall recognition and remembering: a dual-process account. Psychon Bull Rev. September 2001;8(3):587-99.
72: Andersson H, Jonsson C, Moberg C, Stemine G. Patterned self-assembled beads in silicon channels. Electrophoresis. October 2001;22(18):3876-82.
73: Wany, P C, DeVoe D I, Lee C S. Integration of polymeric membranes with microfluidic networks for bioanalytical applications. Electrophoresis. October 2001;22(18):3857-67.
74: Awuah E. Anohene F, Asante K, Lubberding H, Gijzen H. Environmental conditions and pathogen removal in macrophyte- and algal-based domestic wastewater treatment systems. Water Sci Technol. 2001;44(6):11-8.
75: Villavicencio J L, Gillespie D L, Kreishman P. Controlled ischernia for complex venous surgery: The technique of choice. J Vasc Surg. November 2001;34(5):947-951.
76: Rosenthal E, Clark J M, Wax M K, Cook T A. Emerging perceptions of facial plastic surgery among medical students. Otolaryngol Head Neck Surg. November 2001; 125(5):478-82.
77: Kato Y, Moroshima K, Hashizurne M, Ando H, Furukawa M. Further observation of content uniformity of d-alpha-tocopheryl acetate as an oily drug in granules obtained by wet granulation with a high-shear mixer. Drug Dev Ind Pharm. September 2001;27(8):781-7.
78: Wara-aswapati N, Pitiphat W, Chandrapho N, Rattanayatikul C. Karimbux N. Thickness of palatal masticatory mucosa associated with age. J Periodontol. October 2001;72(10):1407-12.
79: Webb L X, Schmidt U. Unfailchirurg. October 2001;104(10):918-26. German.
80: Rolirich R J. Mastering shape and form in cosmetic surgery: the annual meeting of the american society for aesthetic plastic surgery. Plast Reconstr Surg. September 2001; 108(3):741-2. No abstract available.
81: Rurnalla V K, Borah G l. Cytokines, growth factors, and plastic surgery. Plast Reconstr Surg. September 2001; 108(3):719-33.
82: Bradshaw W E, Holzapfel C M. Genetic shift in photoperiodic response correlated with global warming. Proc Natl Acad Sci U S A. Nov. 6, 2001
   References Discussing Polystyrene and Polypropene Materials Useful in the Practice of the Present Invention:
83: Alarcon T, Lopez-Hernandez S, Andreu D, Saugar J M, Rivas L, Lopez-Brea M. In vitro activity of CA(I-8)M(I-18), a synthetic ceeropin A-melittin hybrid peptide, against multiresistant Acinetobacter baumannii strains. Servicio de Microbiologia, Hospital Universitario de la Princesa, Madrid. Rev Esp Quirnioter June 2001; 14(2):184-90
84: Reimann C, Siewers U, Skarphagen H, Banks D. Does bottle type and acid-washing influence trace element analyses by ICP-MS on water samples? A test covering 62 elements and four bottle types: high density polyethene (HDPE), polypropene (PP), fluorinated ethene propene copolymer (FEP) and perfluoroalkoxy polymer (PFA). Sci Total Environ. Oct. 1, 1999;239(1-3):111-30.
85: Onken J, Bemer R G. Biotransformation of citronellol by the basidiomycete Cystoderma carcharias in an aerated-membrane bioreactor. Appl Microbiol Biotechnol. February 1999;51(2):158-63.
86: Alesso S M, Yu Z, Pears Q, Worthinyton P A, Luke R W, Bradley M. Synthesis of Resins via Multiparallel Suspension Polymerization. J Comb Chem. Nov. 12, 2001;3(6):631-633.
87: Rousselot-Pailley P. Ede N J. Lippens G. Monitoring of Solid-Phase Organic Synthesis on Macroscopic Supports by HighResolution Magic Angle Spinning NMR. J Comb Chem. Nov. 12, 2001;3(6):559-563.
88: Green G M, Peet N P, Metz W A. Polystyrene-supported benzenesulfonyl azide: a diazo transfer reagent that is both efficient and safe. J Org Chem. Nov. 16, 2001;66(23):7930. No abstract available.
89: Thiagarajah J R, Jayaraman S, Naftalin R J, Verkman A S In vivo fluorescence measurement of Na(+) concentration in the pericryptal space of mouse descending colon. Am J Physiol Cell Physiol. December 2001;281(6):CI898-903.
90: Ohno K, Azuma Y, Nakano S, Kobayashi T, Hirano S, Nobuhara Y, Yamada T. Assessment of styrene oligomers eluted from polystyrene-made food containers for estrogenic effects in in vitro assays. Food Chem Toxicol. December 2001;39(12):1233-41.
91: Saito T, Akita S, Torii T, Hiraide M. Selective concentration of gold in water to a polystyrene-embedded fiber disk with polyoxyethylene(10)-p-isononylphenyI ether. J Chromatogr A. Oct. 12, 2001;932(1-2):159-63.
92: Verhaea,en F, Palmans H. A systematic Monte Carlo study of secondary electron fluence perturbation in clinical proton beams (70-250 MeV) for cylindrical and spherical ion chambers. Med Phys. October 2001;28(10):2088-95.
93: Barnes K A, Doualas J F, Liu D W, Karim A. Influence of nanoparticles and polymer branching on the dewetting of polymer films. Adv Colloid Interface Sci. Nov. 15, 2001;94(1-3):83-104.
94: Forrest J A, Dalnoki-Veress K. The glass transition in thin polymer films. Adv Colloid Interface Sci. Nov. 15, 2001;94(1-3):167-96.
95: Cai L L, Granick S. Chains end-grafted in a theta-solvent and polymer melts: comparison of forcedistance profiles. Adv Colloid Interface Sci. Nov. 15, 2001;94(1-3):135-50.
96: Mason R, Jalbert C A, O'Rourke Muisener P A, Koberstein J T, Elman, J F, Lon˜, T E, Gunesin B Z. Surface energy and surface composition of end-fluorinated polystyrene. Adv Colloid Interface Sci. Nov. 15, 2001;94(1-3):1-19.
97: Bertanza G, Colliv4znarelli C, Pedrazzani R. The role of chemical oxidation in combined chemical-physical and biological processes: experiences of industrial wastewater treatment. Water Sci Technol. 2001;44(5):109-16.
98: La Barre S, Hamadouche N, El Khadali Z. Gottini Y, Muller D, Erard-Le Denn E, Jozefowicz M. Selective surface adhesion of the toxic microalga Alexandrium minutum induced by contact with substituted polystyrene derivatives. J Biotechnol. Jan. 31, 2002;93(1):59-71.
99: Pu Y, Rafailovich M H, Sokolov J, Gersgppe D, Peterson T. Wu W L, Schwarz S A. Mobility of polymer chains confined at a free surface. Phys Rev Lett. Nov. 12, 2001;87(20):206101.
100: Reiter G. Dewetting of highly elastic thin polymer films. Phys Rev Lett. Oct. 29, 2001;87(18):186 101.
101: Barreiro-Iglesias R, Alvarez-Lorenzo C, Conclieiro A. Incorporation of small quantities of surfactants as a way to improve the rheological and diffusional behavior of carbopol gels. J Control Release. Nov. 9, 2001;77(1-2):59-75.
102: Cho C S, Cho K Y. Park I K, Kim S H, Sasai4awa T, Uchivama M, Akaike T. Receptor-mediated delivery of all trans-retinoic acid to hepatocyte using poly(L-lactic acid) nanoparticles coated with galactose-carrying polystyrene. J Control Release. Nov. 9, 2001;77(1-2):7-15.
103: Kim M C, Kim D S, Lee K W, Youn H J, Choi K B, Ha Y C. Multijet and multistage aerosol concentrator: design and performance analysis. J Aerosol Med. 2001 Summer; 14(2):245-54.
104: Magnuson M L, Lytle D A, Frietch C M, Kelty C A. Characterization of submicrometer aqueous iron(III) colloids formed in the presence of phosphate by sedimentation field flow fractionation with multiangle laser light scattering detection. Anal Chem. Oct. 15, 2001;73(20):4815-20.
   References Discussing Vicryl and Fibrin Materials Useful in the Practice of the Present Invention:
105: Pernot F. Ann Chir. September 2001; 126(7):666-8. French.
106: Sokolov V V, Bagirov M M. Reconstructive surgery for combined tracheo-esophageal injuries and their sequelae. Eur J Cardiothorac Surg. November 2001;20(5):1025-9.
107: Hodgson N C, Malthaner R A, Ostbve T. Current practice of abdominal fascial closure: a survey of Ontario general surgeons. Can J Surg. October 2001;44(5):366-70.
108: Dadeva S, Ms K. Strabismus surgery: Fibrin glue versus vicryl for conjunctival closure. Acta Ophthalmol Scand. October 2001;79(5):515-7.
109: Zhao H, Li Z, Fang J, Fany- C. Circumferential resection and “Z”-shape plastic end-to-end anastomosis of canine trachea. Chin Med J (Engl). March 1999; 112(3):278-80.
110: Tannirandom Y, Tuchinda K. Vaginal vault granulations after total abdominal hysterectomy using polyglactin for vault closure. J Med Assoc Thai. May 2001;84(5):693-6.
111: Sculean A, Berakdar M, Pahl S, Windisch P, Brecx M, Reich E, Donos N. Patterns of cytokeratin expression in monkey and human periodontium following regenerative and conventional periodontal surgery. J Periodontal Res. August 2001;36(4):260-8.
112: Vaidy M, O'Hagan D T. Microparticles for intranasal immunization. Adv Drug Deliv Rev. Sep. 23, 2001;51(1-3):12741. Review.
113: Altarac S, Glavas M, Drazinic 1, Kovac D, Celovic R, Matosevic E, Jerin L. Experimental and clinical study in the treatment of sigmoid volvulus. Acta, Med Croatica. 2001;55(2):67-71.
114: Frings N. Glowacki P, Nelle A, Van-Thanh-Phuonp- T. [Prospective study of avoiding neoangiogenesis after great saphenous vein crossectomy. Initial results]. Zentralbl Chir. July 2001; 126(7):528-30. German.
115: Mallery S R. Shenderova A, Pei P, Begurn S, Ciminieri J R, Wilson R F, Casto B C, Schuller D E, Morse M A. Effects of I O-hydroxycamptothecin, delivered from locally injectable poly(lactide-co-glycolide) microspheres, in a murine human oral squamous cell carcinoma regression model. Anticancer Res. May-June 2001;21(3 13): 1713-22.
116: Lin K Y, Farinholt H M, Reddy V R, Edlich R F, Rodeheaver G T. The scientific basis for selecting surgical sutures. J Long Term Eff Med Implants. 2001;11(1-2):29-40.
117: Laaksovirta S, Talja M, Valimaa T, Isotalo T, Tormala P, Tammela T L. Expansion and bioabsorption of the self-reinforced lactic and glycolic acid copolymer prostatic spiral stent. J Urol. September 2001; 166(3):919-22.
118: Behrend M, Klempnauer J. Influence of suture material and technique on end-to-end reconstruction in tracheal surgery: an experimental study in sheep. Eur Surg Res. May-June 2001;33(3):210-6.
119: Brayden D J, Templeton L, McClean S, Barbour R, Huang, J, Nguyen M, Ahern D, Motter R, Johnsond K, Vasquez N, Schenk D, Seubert P. Encapsulation in biodegradable microparticles enhances serum antibody response to parenterally-delivered beta-amyloid in mice. Vaccine. Jul. 20, 2001; 19(30):4185-93.
120: Straub A M, Suvan J, Lang N P, Mombelli A, Braman V, Massaro J, Friden P, Tonetti M S. Phase I evaluation of a local delivery device releasing silver ions in periodontal pockets: safety, pharmacokinetics and bioavailability. J Periodontal Res. June 2001;36(3):187-93.
121: John T. Pterygiurn excision and conjunctival mini-autograft: preliminary report. Eye. June 2001; I 5(Pt 3):292-6.
122: Ivanoff C J. Widmark G. Nonresorbable versus resorbable sutures in oral implant surgery: a prospective clinical study. Clin Implant Dent Relat Res. 2001;3(1):57-60.
123: Voros A, Ender F, Jakkel T, Cserepes E, Tota J, Szanto I, Ereifej S, Seli A, Farsang Z, Kesseru B, Laszlo S, Polanyi C. [Esophageal anastomosis-based on the experience with 1460 operations]. Magy Seb. June 2001;54(3):132-7. Hungarian.
124: Shen Z L, Berger A, Hierner R, Allmeling C, Ungewickell E, Walter G F. A Schwann cell-seeded intrinsic framework and its satisfactory biocompatibility for a bioartificial nerve graft. Microsurgery. 2001;21(1):6-11.
125: Bezerra C A, Bruschini H. Suburethral sling operations for urinary incontinence in women (Cochrane Review). Cochrane Database Syst Rev. 2001;3:CDO01754.
126: Hunziker E B, Driesaruz I M, Saaaer C. Structural barrier principle for growth factor-based articular cartilage repair. Clin Orthop. October 2001;(391 Suppl): S 182-9.
127: Irshad K, Feldman L S, Lavoie C, Lacroix. V J, Mulder D S, Brown R A. Operative management of “hockey groin syndrome”: 12 years of experience in National Hockey League players. Surgery. October 2001; 130(4):759-66.
128: Agarwal R, McDougal G. Buzz in the axilla: a new physical sign in hemodialysis forearm graft evaluation. Am J Kidney Dis. October 2001;38(4):853-7.
129: Wu Y, Hu B, Peniz T, Jiang Z. In-situ separation of chromium(III) and chromium(VI) and sequential ETV-ICP-AES determination using acetylacetone and PTFE as chemical modifiers. Fresenius J Anal Chem. August 2001;370(7):904-8.
130: Dobrowolski R. Determination of selenium in soils by slurry-sampling graphite-furnace atomic-absorption spectrometry with polytetrafluoroethylene as silica modifier. Fresenius J Anal Chem. August 2001;370(7):850-4.
131: Prager M. Polterauer P, Bohmig H J, Wapner O, Fugl A, Kretschmer G. Plohner M, Nanobashvili J, Huk I. Collagen versus gelatin-coated Dacron versus stretch polytetrafluoroethylene in abdominal aortic bifurcation graft surgery: results of a seven-year prospective, randomized multicenter trial. Surgery. September 2001;130(3):408-14.
132: Chattopadhyay D, Galeska 1, Pgpadimitrakopoulos F. Metal-assisted organization of shortened carbon nanotubes in monolayer and multilayer forest assemblies. J Am Chem Soc. Sep. 26, 2001;123(38):9451-2. No abstract available.
133: Brody H J. Complications of expanded polytetrafluoroethylene (e-PTFE) facial implant. Dermatol Surg. September 2001;27(9):792-4.
134: Pelaez Mata D, Alvarez Zapico J A. [Current aspects in the treatment of vesicoureteral reflux. Analysis of our experience]. Cir Pediatr. July 2001; 14(3):112-5. Spanish.
135: Dixon M A, Grodzinski B, Cote R, Stasiak M. Sealed environment chamber for canopy light interception and trace hydrocarbon analyses. Adv Space Res. 1999;24(3):271-80.
136: Golub M A, Wvdeven T, Johnson A L. On the similarity of plasma-polymerized tetrafluoroethylene and RF plasma-sputtered polytetrafluoroethylene. Polymer Prepr. 1997;38(2):668-9. No abstract available.
137: Ryan K G, Ireland W. A small-scale outdoor plant growth chamber with modulated enhancement of solar UV-B radiation. J Environ Qual. May-June 1997;26(3):866-71.
138: Oberdorster G, Gelein R M. Ferin J, Weiss B. Association of particulate air pollution and acute mortality: involvement of ultrafine particles? Inhal Toxicol. January-February 1995;7(1):111-24.
139: Golub M A. Concerning apparent similarity of structures of fluoropolymer surfaces exposed to an argon plasma or argon ion beam. Langmuir. 1996; 12(13):3360-1. No abstract available.
140: Schmidt P, Felliner J. Hahn T. Hubner K. Investigation of the low-energy component of primary cosmic radiation on the outside of spacecrafts. Adv Space Res. October 1994; 14(10):61-5.
141: Rashid S N, Clark H G, Vann R D, Gertli W A, Palmos L A, Mikat E M. The effect of interstitial air on the in vitro thrombogenicity of ePTFE vascular grafts. J Bioact Compat Polyrn. January 1992;7(1):54-64.
142: Todd P, Sklar V, Ramirez W F, Smith G J, Moraenthaler G W, McKinnon J T, Oberdorster G, Schulz J. Inhalation risk in low-gravity spacecraft. Acta Astronaut. July 1994;33:305-15.
143: Ferin J, Oberdorster G. Polymer degradation and ultrafine particles: potential inhalation hazards for astronauts. Acta Astronaut. 1992;27:257-9. PMID: 11537570 [PubMed—indexed for MEDLINE]
   References Discussing Polyactide Materials Useful in the Practice of the Present Invention:
144: Braiidoii H J, Young V L, Jerina K L, Wolf C J. Variability in the properties of silicone gel breast implants. Plast Reconstr Surg. September 2001;108(3):647-55.
145: Leatherman B D, Dornhoffer J L, Fan C Y, Mukunyadzi P. Dermineralized bone matrix as an alternative for mastoid obliteration and posterior canal wall reconstruction: results in an animal model. Otol Neurotol. November 2001;22(6):731-6.
146: Jukkala-Partio K, PohJonen L Laitinen O, Partio E K, Vasenius J, Toivonen T, Kinnunen J, Tormala P, Rokkanen P. Biodegradation and strength retention of poly-L-lactide screws in vivo. An experimental long-term study in sheep. Ann Chir Gynaecol. 2001;90(3):219-24.
147: Geurs N C. Wang I C, Shulman L B, Jeffcoat M K. Retrospective radiographic analysis of sinus graft and implant placement procedures from the academy of osseointegration consensus conference on sinus grafts. Int J Periodontics Restorative Dent. October 2001;21(5):517-23.
148: Bahat O, Fontanesi F V. Complications of grafting in the atrophic edentulous or partially edentulous jaw. Int J Periodontics Restorative Dent. October 2001;21(5):487-95.
149: Weihe S. Wehmoller M, Tschakaloff A. von Oepen R, Schiller C, Epple M, Etifinger H. Mund Kiefer Gesichtschir. September 2001;5(5):299-304. German.
150: Ragde H, Grado G L, Nadir B S. Brachytherapy for clinically localized prostate cancer: thirteen-year disease-free survival of 769 consecutive prostate cancer patients treated with permanent implants alone. Arch Esp Urol. September 2001;54(7):739-47.
151: Narcisi E M. Three-unit bridge construction in anterior single-pontic areas using a metal-free restorative. Compend Contin Educ Dent. February 1999;20(2):109-12, 114, 116-9; quiz 120.
152: Verheggen R, Merten H A. Correction of Skull Defects Using Hydroxyapatite Cement (HAC)—Evidence Derived from Animal Experiments and Clinical Experience. Acta Neurochir (Wicn). September 2001; 143(9):919-26.
153: Bonnomet F, Vanhille W, Lefebvre Y, Clavert P, Gicquel P. Kempf J F. [Failure of acetabular cups fixed with a cement armed with a thick embedded wire mesh). Rev Chir Orthop Reparatrice Appar Mot. October 2001;87(6):544-55. French.
154: Kim E S, Park E J, Choung P H. Platelet concentration and its effect on bone formation in calvarial defects: An experimental study in rabbits. J Prosthet Dent. October 2001;86(4):428-33.
155: Futran N D. Retrospective case series of primary and secondary microvascular free tissue transfer reconstruction of midfacial defects. J Prosthet Dent. October 2001;86(4):369-76.
156: Spreafico C, Patelli G, Lanocita R, Di Tolla G, Marchiano A, Frigerio L, Garbagiiati F, Ticha V, Dainascelli B. Percutancous implant of Denver peritoneovenous shunt: a new opportunity for the interventional radiologist. Radiol Med (Torino). September 2001; 102(3):154-8.
157: Bellelli A. Avitto A, Liberali M, lannetti F, lannetti L, David V. Osteo-odonto-kerato-prothesis. Radiographic, CT and MR features. Radiol Med (Torino). September 2001; 102(3):143-7.
158: Gaggl A, Schultes G, Karcher H. Navigational precision of drilling tools preventing damage to the mandibular canal. J Craniomaxillotac Surg. October 2001;29(5):271-5.
159: Proussaefs P, Lozada J. Histologic evaluation of a 9-year-old hydroxyapatite-coated cylindric implant placed in conjunction with a subantral augmentation procedure: a case report. Int J Oral Maxillofac Implants. September-October 2001; 16(5):737-41.
160: Szabo G, Suba Z, Hrabak K, Barabas J, Nemeth Z. Autogenous bone versus beta-tricalcium phosphate graft alone for bilateral sinus elevations (2- and 3-dimensional computed tomographic, histologic, and histomorphometric evaluations): preliminary results. Int J Oral Maxillofac Implants. September-October 2001; 16(5):681-92.
161: Glickman R S, Bae R, Karl is V. A model to evaluate bone substitutes for immediate implant placement. Implant Dent. 2001;10(3):209-15.
162: Novaes A B Jr, Souza S L. Acellular dermal matrix graft as a membrane for guided bone regeneration: a case report. Implant Dent. 2001; 10(3):192-6.
163: Zusman R, Zusman I. Glass fibers covered with sol-gel glass as a new support for affinity chromatography columns: a review. J Biochem Biophys Methods. Oct. 30, 2001;49(1-3):175-87.
164: John J, Gaiwadhar S A, Shah I. Flexural strength of heat-polymerized polymethyl methacrylate denture resin reinforced with glass, aramid, or nylon fibers. J Prosthet Dent. October 2001;86(4):424-7.
165: Marsh G M, Youk A O, Stone R A, Buchanich J M, Gula M J, Smith T j. Quinn N M. Historical cohort study of US man-made vitreous fiber production workers: I. 1992 fiberglass cohort follow-up: initial findings. J Occup Environ Med. September 2001;43(9):741-56.
166: Narva K K Vallittu P K Helenius H Yli-Urpo A. Clinical survey of acrylic resin removable denture repairs with glass-fiber reinforcement. Int J Prosthodont. May-June 2001;14(3):219-24.
167: Kawano F, Kon M, Kobayashi M, Miyai K. Reinforcement effect of short glass fibers with CaO—P(2)0(5) —SiO(2) AI(2)0(3) glass on strength of glass-ionomer cement. J Dent. July 2001;29(5):377-80.
168: Heiner A D, Brown T D. Structural properties of a new design of composite replicate femurs and tibias. J Biomech. June 2001;34(6):773-81.
169: Heiner A D, Brown T D. Morphology of glass fibers in electronics workers with fiberglass dermatitis—a scanning electron microscopy study. Int J Dermatol. April 2001;40(4):258-61.
170: Kamstrup O, Ellehau(ge A, Chevalier J, Davis J M. McConnell E F. Thevenaz P. Chronic inhalation studies of two types of stone wool fibers in rats. Inhal Toxicol. July 2001; 13(7):603-21.
171: Lassila L V, Vallittu P K. Denture base polymer Alldent Sinomer: mechanical properties, water sorption and release of residual compounds. J Oral Rehabil. July 2001;28(7):607-13.
172: Quintas A F, Dinato J C, Bottino M A. Aesthetic posts and cores for metal-free restoration of endodontically treated teeth. Pract Periodontics Aesthet Dent. November-December 2000; 12(9):875-84; quiz 886.
173: Martelli R. Fourth-generation intraradicular posts for the aesthetic restoration of anterior teeth. Pract Periodontics Aesthet Dent. August 2000; 12(6):579-84; quiz 586-8.
174: Adams T. Restoration of an endodontically treated tooth utilizing a single-unit crown and core system. Pract Periodontics Aesthet Dent. January-February 2000; 12(1):105-8. No abstract available.
175: Cormier C J, Burns D R, Moon P. In vitro comparison of the fracture resistance and failure mode of fiber, ceramic, and conventional post systems at various stages of restoration. J Prosthodont. March 2001; 10(1):26-36.
176: Goldberg, M S, Parent M E, Siemiatvcki J, Desy M, Nadon L, Richardson L, Lakhani R, Latreille B, Valois N T F. A case-control study of the relationship between the risk of colon cancer in men and exposures to occupational agents. Am J Ind Med. June 2001;39(6):531-46.
177: Tanner J, Vallittu P K, Soderling E. Effect of water storage of E-glass fiber-reinforced composite on adhesion of Streptococcus mutans. Biomaterials. June 2001;22(12):1613-8.
178: Nagai E, Otani K, Satoh Y, Suzuki S. Repair of denture base resin using woven metal and glass fiber: effect of methylene chloride pretreatment. J Prosthet Dent. May 2001;85(5):496-500.
179: Nagai E, Otani K, Satoh Y, Suzuki S. Estimation of fibrous aerosol deposition in upper bronchi based on experimental data with model bifurcation. Ind Health. April 2001;39(2):141-9.
180: Uzun G, Keyf F. The effect of woven, chopped and longitudinal glass fibers reinforcement on the transverse strength of a repair resin. J Biomater Appl. April 2001; 15(4):351-8.
181: Swauger J E. Correspondence re: Cummings et al., Consumer perception of risk associated with filters contaminated with glass fibers. Cancer Epiderniol. Biomark. Prev., 9: 977-979, 2000. Cancer Epiderniol Biomarkers Prev. April 2001; 10(4):416-8. No abstract available.
182: Breysse P N, Lees P S, Rooney B C, McArthur B R, Miller M E, Robbins C. Breysse P N, Lees PS, Rooney B C, McArthur B R, Miller M E, Robbins C. End-user exposures to synthetic vitreous fibers: II. Fabrication and installation fabrication of commercial products. Appl Occup Environ Hyg. April 2001; 16(4):464-70.
   References Discussing Dacron Materials Useful in the Practice of the Present Invention:
183: Toursarkissian B, Smilanich R P, Sykes M T. Related Articles Autologous superficial femoral vein for the repair of suprarenal mycotic aneurysms: a preferred conduit?: a case report. Vasc Surg. March-April 2001;35(2):157-61.
184: Vega D, Polo J R, Polo J, Lopez Baena J A, Pacheco D, Garcia-Pajares R. Related Articles Brachial-jugular expanded PTFE grafts for dialysis. Ann Vasc Surg. September 2001; 15(5):553-6.
185: Bickels J, Wittig J C, Kollender Y, Neff R S, Kellar-Graney K, Meller I, Malawer M M. Related Articles Reconstruction of the extensor mechanism after proximal tibia endoprosthetic replacement. J Arthroplasty. October 2001; 16(7):856-62.
186: Vermculen F, Schepens M, de Valois J, Wijers L, Kelder J. Related Articles The Hemashield Woven((R)) prosthesis in the thoracic aorta: a prospective computer tomography follow-up study. Cardiovasc Surg. December 2001;9(6):580-5.
187: Manganas C, Iliopoulos J, Chard R B, Nunn G R. Related Articles Reoperation and coarctation of the aorta: the need for lifelong surveillance. Ann Thorac Surg. October 2001;72(4):1222-4.
188: Auguste K I, Quinones-Hincjosa A, Lawton M T. Related Articles The tandem bypass: subclavian artery-to-middle cerebral artery bypass with dacron and saphenous vein grafts. Technical case report. Surg Neurol. September 2001;56(3):164-9.
189: Decker P, Born M, Decker D, Hirner A. Related Articles Chirurg. September 2001;72(9):1067-70. German.
190: Rosen M, Grossman E S, Cleaton-Jones P E, Volchansky A. Related Articles Surface roughness of aesthetic restorative materials: an in vitro comparison. SADJ. July 2001;56(7):316-20.
191: Shigematsu K, Shigematsu H, Nishikage S, Origuchi N, Ishikawa 1, Furuta Y. Related Articles Non-anastomotic midgrafts stenosis of a knitted Dacron graft after arterial reconstruction. Report of a case. Int Angiol. September 2001;20(3):248-50.
192: Jetten J, de Kruijf N, Castle L. Related Articles Quality and safety aspects of reusable plastic food packaging materials: a European study to underpin future legislation. Food Addit Contam. January 1999; 16(1):25-36.
193: Cruz C P, Drouilhet J C, Southern F N, Eidt J F, Barnes R W, Moursi M M. Related Articles Abdominal aortic aneurysm repair. Vasc Surg. September-October 2001;35(5):335-44. Review.
194: Prager M, Polterauer P, Bohmig H J, Wagner O, Fugl A, Kretschmer G, Plohner M, Nanobashvili J, Huk I. Related Articles Collagen versus gelatin-coated Dacron versus stretch polytetrafluoroethylene in abdominal aortic bifurcation graft surgery: results of a seven-year prospective, randomized multicenter trial. Surgery. September 2001; 130(3):408-14.
195: Akashi H, Tayama K, Fujino T, Hayashi S, Tobinaga S, Aoyagi S. Related Articles Unruptured aneurysm of the right coronary sinus of Valsalva with type B aortic dissection. J Cardiovasc Surg (Torino). October 2001;42(5):625-7.
196: Vollmann D, Ruschewski W, Unterberg C. Related Articles Aortic recoarctation as the source of arterial embolism 32 years after synthetic patch angioplasty. Heart. October 2001;86(4):410. No abstract available.
197: Shinonaga M, Kanazawa H, Nakazawa S, Yoshiya K, Yamazaki Y. Related Articles [Total arch replacement following partial replacement of the descending aorta for acute type A aortic dissection: report of a case]. Kyobu Geka. September 2001;54(10):825-8. Japanese.
198: Santhosh Kumar T R, Krishnan L K. Related Articles Endothelial cell growth factor (ECGF) enmeshed with fibrin matrix enhances proliferation of EC in vitro. Biomaterials. October 2001;22(20):2769-76.
199: Waheed A, Majeed A, Cera F, Tiveron P, Cherubini R, Moschini G, Khan E U. Related Articles Use of track detectors in biomedical sciences. Nucl Tracks Radiat Meas. 1993;22(1-4):889-92.
200: Miller D P, Howell G S, Fiore J A. Related Articles A whole-plant, open, gas-exchange system for measuring net photosynthesis of potted woody plants. Hort Science. October 1996;31(6):944-6.
201: Kahn B A, Stoffella P J. No evidence of adverse effects on germination, emergence, and fruit yield due to space exposure of tomato seeds. J Am Soc Hortic Sci. May 1996; 121(3):414-8.
202. Redmond, Eileen M., Cahill, Paul A., Sitzmann, James V. Perfused Transcapillary Smooth Muscle Cells and Endothelial Cell Co-Culture—A Novel In Vitro Model. In Vitro Cellular and Developmental Biology—Animal 31:601-609, September 1995.

1. An artificial vessel scaffold, comprising:
a plurality of elongated scaffold panels arranged in laterally abutting relation to form a tubular structure; and
a plurality of circular fibers,
wherein said elongated scaffold panels each comprise a first parallel strand of fiber and a second parallel strand of fiber fixedly connected by a plurality of connection fibers oriented substantially perpendicular to said first parallel strand and said second parallel strand,
wherein said circular fibers encircle and are fixedly connected to said tubular structure, and
wherein said tubular structure defines an inner diameter and an outer diameter.

2. The artificial vessel scaffold of claim 1, further comprising a layer of endothelial cells attached to said internal diameter of the artificial vessel scaffold.

3. The artificial vessel scaffold of claim 2, further comprising a layer of smooth muscle cells attached to said outer diameter of the artificial vessel scaffold.

4. The artificial vessel scaffold of claim 3, wherein the artificial vessel has an inner diameter of 0.5 to 3.0 cm.

5. The artificial vessel scaffold of claim 1, further comprising a layer of digestible material within said internal diameter and attached to the artificial vessel scaffold.

6. A cellular growth chamber, comprising:
a vessel;
an opening in said vessel that allows insertion and removal of a vessel scaffold and that is sealingly closeable;
a sealable port providing a opening in said vessel and allowing inlet and outlet of cell culture solution; and
an environmental control capable of monitoring environmental conditions within said vessel.

7. The cellular growth chamber of claim 6, wherein said sealable port comprises a first port with an inlet tube and a second port with an outlet tube.

8. The cellular growth chamber of claim 6, wherein said environmental control provides adjustment of environmental conditions for favorable cell growth conditions.

9. An artificial vessel scaffold coated with cellular material in a cellular growth chamber according to claim 6.

10. An artificial organ comprising a plurality of artificial vessel scaffolds according to claim 1.

11. An artificial organ comprising a plurality of artificial vessel scaffolds coated with cells in a device according to claim 6.

12. An artificial liver, comprising:
a common entry region comprised of a hollow cylindrical tube and having a first end and a second end;
at least four individual entry regions each having a first end and a second end, said first end of said individual entry regions being fixedly connected to said second end of said common entry;
at least four inner vessels, said inner vessels having a first end and a second end, said first end of said inner vessels being fixedly connected to said second end of said individual entry regions;
at least four individual exit regions having a first end and a second end, said first end of said individual exit portions being fixedly connected to said second end of said inner vessels; and
a common exit region comprised of a hollow cylindrical tube and having a first end and a second end, said first end of said common exit region being fixedly connected to said second end of said individual exit regions,
wherein said first end of said common entry portion and said second end of said common exit portions are fixedly connected to a patient,
wherein said individual entry regions, said inner vessels, and said individual exit regions are comprised of an artificial scaffold having an inner surface and an outer surface,
wherein said inner surface of said artificial scaffold is coated with vascular endothelial cells, and
wherein said outer surface of said artificial scaffold is coated with hepatocytes.

13. An external artificial liver, comprising:
an artificial liver according to claim 12 located within a waterproof container having a first end and a second end,
a first pump located at said first end of said container; and
a second pump located at said second end of said container,
wherein said first end of said waterproof container is connected to and in liquid communication with a patient's artery, and
wherein said second end of said waterproof container is connected to and in liquid communication with said patient's vein.

14. An internal artificial liver, comprising:
an artificial liver according to claim 12 located within a waterproof container having a first end and a second end,
a first pump located at said first end of said container; and
a second pump located at said second end of said container,
wherein said first end of said waterproof container is connected to and in liquid communication with a patient's artery, and
wherein said second end of said waterproof container is connected to and in liquid communication with said patient's vein.

15. An artificial pancreas, comprising:
at least two artificial pancreas units, each unit comprising a cylindrical mainline scaffold and a plurality of side branches and having a first end and a second end;
a first connection tube having a first end fixedly connected to said first end of said artificial pancreas units and having a second end fixedly connected to and in fluid communication with a patient; and
a second connection tube having a first end fixedly connected to said second end of said artificial pancreas units and having a second end fixedly connected to and in fluid communication with a patient,
wherein said mainline scaffold has a first end and a second end,
wherein said side branches have a first end and a second end,
wherein said first end of said side branches are fixedly connected and in fluid communication with said mainline scaffold,
wherein said second end of said side branches are fixedly connected and in fluid communication with said mainline scaffold at a point on said mainline scaffold closer to said second end of said mainline scaffold than said first end of said mainline scaffold, and
wherein said mainline scaffold and said side branches are coated with hormone producing islet cells.

16. An artificial heart valve, comprising:
a circular ring; and
a plurality of leaflets each having two generally parallel generally flat surfaces and an edge around the perimeter of said leaflets,
wherein a first portion of said leaflet edge is fixedly and flexibly connected to said circular ring,
wherein said leaflet is sized and shaped so a second portion of said leaflet edge opposite said first portion of said leaflet edge is located approximately in the center of said circular ring, and
wherein said circular ring and said leaflets are comprised of a scaffold according to claim 1.

17. An artificial cardiac ventricle, comprising:
a hollow generally cylindrical central region having a bottom and an apex, said bottom being wider than said apex; and
a hemispheric base region fixedly connected to said bottom of said central region,
wherein said central region and said base region are comprised of a scaffold according to claim 1.

18. The artificial cardiac ventricle of claim 16, further comprising:
a jacket enveloping the artificial cardiac ventricle and fixedly attached to said apex of said central region.

19. A cardiac pump, comprising:
a motor in communication with a pumping means; and
a generally cylindrical compressible cardiac replacement unit,
wherein said pumping means is comprised of at least one wheel capable of rolling along the length of said cardiac replacement unit to compress and decompress said cardiac replacement unit.

20. A cardiac pump, comprising:
a motor in communication with a pumping means; and
a generally cylindrical compressible cardiac replacement unit located inside a fluid displacement unit and in fluid communication with at least one tube leading outside said fluid displacement unit,
wherein said pumping means is comprised of a fluid displacement device, said fluid displacement device being capable of increasing and decreasing the pressure of fluid within said fluid containment unit, and
wherein said increasing and decreasing pressure of fluid causes compression and decompression of said cardiac replacement unit.

21. An artificial cardiac device, comprising:
two artificial cardiac ventricles, each comprising
a hollow generally cylindrical central region having a bottom and an apex, said bottom being wider than said apex;
a hemispheric base region fixedly connected to said bottom of said central region; and
at least one cardiac pump according to claim 19,
wherein said at least one pump compresses and decompresses said two artificial cardiac ventricles.

22. The artificial cardiac device of claim 21, further comprising:
a biocompatible housing located around the outer boundaries of said cardiac device, wherein said biocompatible housing containing said cardiac device is located inside a patient.

23. An artificial organ scaffold, comprising:
a porous polymeric material, comprising at least one water-insoluble polymer and at least one water-soluble polymer,
wherein said scaffold has a porosity from approximately 50 to 80% and a pore size from approximately 0.5 micron to 5.0 microns.

24. The artificial organ scaffold of claim 23, wherein said water-insoluble polymer comprises nylon-11.

25. The artificial organ scaffold of claim 23, wherein said water-insoluble polymer comprises nylon-11, and said water-soluble polymer comprises polyethylene oxide.

26. The artificial organ scaffold of claim 25, further comprising calcium carbonate.

27. The artificial organ scaffold of claim 26, wherein nylon-11 comprises approximately 0.26 weight percent, and polyethylene oxide comprises 0.57 weight percent, and calcium carbonate comprises approximately 0.18 weight percent.

28. An artificial organ, comprising:
a porous polymeric material, comprising at least one water-insoluble polymer and at least one water-soluble polymer, forming a tube shaped scaffold with an inner surface and an outer surface;
wherein said scaffold has a porosity from approximately 50 to 80% and a pore size from approximately 0.5 micron to 5.0 microns;
at least one cell layer on said scaffold;
wherein said cell layer on said scaffold forms an artificial organ.

29. The artificial organ of claim 28, wherein said at least one cell layer comprises endothelial cells.

30. The artificial organ of claim 29, wherein said at least one cell layer further comprises smooth muscle cells.

31. The artificial organ of claim 30, wherein said endothelial cells are on the inner surface of the tube, and said smooth muscle cells are on the outer surface of the tube.

32. A method of making an artificial organ, comprising:
selecting a porous polymeric material, wherein said polymeric material initially comprises at least one water-insoluble polymer and at least one water-soluble polymer;
forming a tube shaped scaffold with an inner surface and an outer surface, wherein said scaffold has a porosity from approximately 50 to 80% and a pore size from approximately 0.5 micron to 5.0 microns, and said porosity is achieved by selective dissolution;
placing said scaffold in a bioreactor;
adding at least one of smooth muscle cells and endothelial cells to said bioreactor; and
growing said cells in said bioreactor until a layer of cells have formed on the surfaces of said scaffold.

33. The method according to claim 32, further comprising applying longitudinal flow to said scaffold in said bioreactor.

34. The method according to claim 32, wherein said at least one water-insoluble polymer comprises nylon-11.

35. The method according to claim 32, wherein said at least one water-soluble polymer comprises polyethylene oxide.

36. The method according to claim 32, wherein said at least one water-insoluble polymer comprises nylon-11, and said at least one water-soluble polymer comprises polyethylene oxide, and wherein said porous polymeric material further comprises initially calcium carbonate.

37. The method according to claim 36, wherein said artificial organ is a vessel.

38. An artificial cardiac device, comprising:
two artificial cardiac ventricles, each comprising
a hollow generally cylindrical central region having a bottom and an apex, said bottom being wider than said apex;
a hemispheric base region fixedly connected to said bottom of said central region; and
at least one cardiac pump according to claim 20,
wherein said at least one pump compresses and decompresses said two artificial cardiac ventricles.

39. The artificial cardiac device of claim 38, further comprising:
a biocompatible housing located around the outer boundaries of said cardiac device, wherein said biocompatible housing containing said cardiac device is located inside a patient.
